(19) United States
(12) Patent Application Publication
(10) Pub. No.: US 2022/0244275 A1
(43) Pub. Date: Aug. 4, 2022

(54) NEURODEGENERATIVE DISEASE THERAPIES UTILIZING THE SKIN-BRAIN AXIS

(71) Applicant: Ohio State Innovation Foundation, Columbus, OH (US)

(72) Inventors: Daniel Gallego-Perez, Columbus, OH (US); Natalia Higuita-Castro, Columbus, OH (US); William Lawrence, Columbus, OH (US); Diego Alzate Correa, Columbus, OH (US)

(21) Appl. No.: 17/622,048

(22) PCT Filed: Jul. 2, 2020
(86) PCT No.: PCT/US2020/040721

§ 371 Date: Dec. 22, 2021

Related U.S. Application Data
(60) Provisional application No. 62/869,788, filed on Jul. 2, 2019.

(51) Int. Cl. G01N 33/68; A61K 31/713

(52) U.S. Cl. CPC G01N 33/6896; G01N 2800/2821; A61K 31/713

(57) ABSTRACT

As disclosed herein, the skin can dispatch signals to the brain in the form of exosomes, referred to herein as a “skin-brain axis.” Therefore, disclosed herein is a method for diagnosing a brain disease, disorder, or injury in a subject that involves isolating exosomes from the subject and assaying the exosomes for the presence of one or more biomarkers of the disease, disorder, or injury. Also disclosed are methods of treating a subject with a brain disease, disorder, or injury that involves engineering the skin of the subject to produce therapeutic exosomes. Also disclosed are methods of collecting skin-produced exosomes and loading them with therapeutic cargo that can treat one or more diseases, disorders, or injuries of the brain. Also disclosed herein is a method to reduce exosomal release from the skin to reducing trafficking to the brain.

### CROSS-REFERENCE TO RELATED APPLICATIONS

This application claims benefit of U.S. Provisional Application No. 62/869,788, filed Jul. 2, 2019, which is hereby incorporated herein by reference in its entirety.

### BACKGROUND

Amyloidosis refers to a group of pathologies characterized by the toxic misfolding of proteins into β-pleated sheet fibrils leading to either a systemic or local aggregation and deposition in specific organs or tissues (Merlini, G. & Bellotti, V. N. Engl. J. Med. 2003 349:583-596). Numerous proteins and peptides forming amyloid deposits are associated with multiple human diseases targeting several organs (Chiti, F. & Dobson, C M. Annu. Rev. Biochem. 2017 86:27-68). For instance, neurodegenerative diseases such as Alzheimer's Disease (AD), Parkinson's disease (PD), amyotrophic lateral sclerosis (ALS), and Huntington's disease (HD), are all considered local amyloidosis in which characteristic pathogenic amyloid proteins aggregate in the brain (Tillement, J.-P., et al. Pharmacology 2010 85:1-17).

Alzheimer's Disease (AD) is a neurodegenerative disease characterized by memory loss and a progressive detriment of cognitive and executive functions and constituting the most common form of dementia, currently affecting more than 50 million people worldwide (Alzheimers. Dement. 2020 doi:10.1002/alz.12068). Accumulating evidence establishes a causal relationship between neuronal cell loss and the accumulation of neuropathological lesions formed by amyloid-beta (Aβ) plaques and phosphorylated Tau tangles inside AD-affected brains (Small, S A. & Duff, K. Neuron 2008 60:534-542).

### SUMMARY

As disclosed herein, the skin can dispatch signals to the brain in the form of exosomes, and exosomes derived from the skin of Alzheimer's Disease (AD) subjects contain neurotoxic cargo that could potentially be impacting the progression of this disease. This is a paradigm-shifting concept in AD, referred to herein as a “skin-brain axis.”

In some embodiments, exosomes derived from the skin can be cumulatively carrying neurotoxic cargo to the brain and contributing to the onset and/or progression of neurodegenerative diseases such as Alzheimer's Disease (AD), Parkinson's disease (PD), amyotrophic lateral sclerosis (ALS), and Huntington's disease (HD) (i.e., skin-brain axis).

Therefore, disclosed herein is a method for diagnosing a neurodegenerative disease in a subject that involves isolating exosomes from the subject and assaying the exosomes for the presence of one or more biomarkers. In some embodiments, the method involves assaying the exosomes for the regulation of one or more pathways related to neurodegenerative disease. For example, the method can involve assaying for dysregulation of any pathway associated with a brain disorder, disease, or injury.

Moreover, as disclosed herein exosome engineering approaches can be used as a therapeutic strategy for the brain, using the skin as a “window” to the brain. In some embodiments, the disclosed compositions and methods can be used to treat any injury, disease, or disorder of the brain by delivering a therapeutic gene or cargo. In some embodiments, the brain disease is a neurodegenerative disease such as Alzheimer's Disease (AD), Parkinson's disease (PD), amyotrophic lateral sclerosis (ALS), and Huntington's disease (HD). In some embodiments, the brain disease is a brain cancer, including primary and secondary brain cancers. In some embodiments, the brain disease or injury involves a stroke or ischemia. In some embodiments, the brain injury is a traumatic brain injury.

Therefore, disclosed herein are methods of treating a subject that involves engineering the skin of the subject to produce therapeutic exosomes.

Also disclosed are methods of collecting skin-produced exosomes and loading them with therapeutic cargo, such as those described herein that can treat one or more diseases, disorders, or injuries of the brain.

Also disclosed herein is a method to reduce exosomal release from the skin to reducing trafficking to the brain. For example, in some embodiments, neutral sphingomyelinase inhibitor GW4869 can be applied topically and/or via intradermal injection to reduce skin-exosome release.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows proposed skin-brain axis and its role in delivering neurotoxic cargo to the brain via exosomes, contributing to the development and/or progression of AD.

FIG. 2A shows TNT used to deliver CD63-GFP plasmids to the skin of healthy mice to label the exosomes. Control skin was TNT-treated with mock/empty plasmids. FIG. 2B shows GFP signal detected in the brain 24 hours post-TNT on the skin (absent in control), suggesting that skin-derived exosomes appeared to be able to enter circulation and lodge in the brain.

FIG. 3A shows qRT-PCR data of the relative expression of a mutated human version of amyloid precursor protein (hAPP) mRNA only seen in AD vs. healthy mice. FIG. 3B shows immunoblot analysis of amyloid-beta protein in skin-derived exosomes from healthy and AD mice confirming the presence only under AD.

FIGS. 4A to 4C show differential expression and IPA ontology analysis on RNA content of skin EVs. Volcano plots presenting differential gene content in EVs (Top) and Canonical pathways enriched by differential gene content (Bottom) of 3×Tg-AD compared with B6129SF2/J at 23 weeks (FIG. 4A); B6129SF2/J and 3×Tg-AD at 23 weeks compared with 10 weeks (FIG. 4B); 3×Tg-AD 23 weeks compared with B6129SF2/J 10 weeks, and 3×Tg-AD 10 weeks compared with B6129SF2/J 23 weeks (FIG. 4C). For canonical pathway analysis, blue bars represent pathway downregulation (z-score <0), white bars represent (z-score=0), and gray bars represent no pattern of regulation.

FIGS. 5A and 5B shows skin-derived EVs from 3×Tg-AD mice contain mRNA of transgenic genes for human APP and MAPT. Absolute qPCR data of skin-derived exosomes from B6129SF2/J and 3×Tg-AD mice indicating the presence of transgenic human APP (FIG. 5A) and human MAPT (FIG. 5B) in 3×Tg-AD EVs.

FIGS. 6A and 6B show qPCR data of primary neuron cultures exposed to 3×Tg-AD and B6129SF2/J skin-derived EVs for 24 hours measuring expression of transgenic hAPP (FIG. 6A) and hMAPT (FIG. 6B). mRNA of transgenic hAPP and hMAPT was found in murine primary embryonic neurons exposed to skin-derived EVs from 3×Tg-AD mice and absent and controls.

FIGS. 7A to 7C show 60× deconvolution (FIG. 7A) and confocal images (FIG. 7B) of skin-derived EVs from 3×Tg-AD mice that were fluorescently labeled using PKH26 Red Fluorescent Cell Linker Kit (Sigma). The fluorescently labeled EVs were exposed to murine primary neuron cultures, followed by ICC with neuron specific TUJ1 and stained with DAPI. FIG. 7C shows resulting quantification of immunofluorescence data shows labeled EVs in and around TUJ1⁺ cells at significantly greater levels than TUJ1⁻.

FIGS. 8A and 8B show Live/Dead cell viability assay of murine primary embryonic neuron cultures exposed to 3×Tg-AD/B6129SF2/J skin-derived EVs. FIG. 8A shows 20× fluorescent images of live/dead cell viability assay in neurons. FIG. 8B shows quantification data of dead cells per square millimeter.

FIGS. 9A and 9B show differential expression analysis on RNA content of skin EVs. Volcano and heat map plots presenting differential gene content in Evs of 3×Tg-AD compared with B6129SF2/J at 23 weeks (A) 3×Tg-AD 23 weeks compared with with B6129SF2/J 23 weeks and 3×Tg-AD 10 weeks compared with B6129SF2/J 10 weeks (B) B6129SF2/J 23 weeks compared with B6129SF2/J 10 weeks and 3×Tg-AD 23 weeks compared with 3×Tg-AD 10 weeks.

FIGS. 10A and 10B show Top Canonical Pathways (FIG. 10A) and Disease/Functions Pathways (FIG. 10B) among all differential expression comparison groups show multiple dysregulated pathways in 3×Tg-AD mice compared with controls.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W. H. Freeman and Company, New York (1993); Posttranslational Covalent Modification of Proteins, B. C. Johnson, Ed., Academic Press, New York, pp. 1-12 (1983)).

As used herein, the term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

A “nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software.

By “specifically hybridizes” is meant that a probe, primer, or oligonucleotide recognizes and physically interacts (that is, base-pairs) with a substantially complementary nucleic acid (for example, a c-met nucleic acid) under high stringency conditions, and does not substantially base pair with other nucleic acids.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

The “control elements” or “regulatory sequences” are those non-translated regions of the vector—enhancers, promoters, 5′ and 3′ untranslated regions—which interact with host cellular proteins to carry out transcription and translation. Such elements may vary in their strength and specificity.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

### Skin-Brain Axis

Alzheimer's Disease (AD) hallmark proteins, Amyloid Precursor Protein (APP), the AR peptide, and Tau are produced in multiple tissues outside the nervous system, suggesting that AD may be a systemic disease involving multiple organs. Outside the nervous system, amyloidosis has also been observed in the skin, the largest organ of the body (Feito-Rodriguez, M. et al. Actas Dermo-Sifiliográficas (English Edition) 2008 99:648-652), in which both Aβ peptides and Tau proteins are produced and amyloid aggregations are formed (Akerman, S C. et al. J. Alzheimers. Dis. 2019 69:463-478; Dugger, B. N. et al. J. Alzheimers. Dis. 2016 51:345-356). Furthermore, AR peptide and its oligomers are found in the brain and in the endothelium of blood vessels in the skin of AD patients and healthy subjects (Kucheryavykh, L Y., et al. Int. J. Mol. Sci. 2018 19), and alterations in skin physiology are commonly observed in AD patients (Schreml, S., et al Exp. Dermatol. 2010 19:953-957). These alterations include metabolic changes (Jong, Y J I., et al. FASEB J. 2003 17:2319-2321; Pani, A. et al. J. Alzheimers. Dis. 2009 18:829-841; Pitto, M. et al. Neurobiol. Aging 2005 26:833-838), changes in calcium metabolism and proliferation (Peterson, C., Proc. Natl. Acad. Sci. U.S.A 1986 83:7999-8001), and reduction in vascular function (Kalman, J. et al. Int. J. Geriatr. Psychiatry 2002 17:371-374), indicating the existence of a skin-brain axis connected through physiological and pathological mechanisms (Clos, A L., et al. Front. Neurol. 2012 3:5).

Previous findings indicate that circulating AR peptides synthesized in peripheral cells are able to cross the blood-brain barrier (Zlokovic, B V. et al. Biophys. Res. Commun. 1994 205:1431-1437), initiate Aβ-dependent neuropathologies and induce neuronal damage (Bu, X.-L. et al. Mol. Psychiatry 2018 23:1948-1956) through a prion-like mechanism (Jucker, M. & Walker, L C. Nature 2013 501:45-51). Moreover, another set of studies demonstrate that APP processing occurs intracellularly via the endocytic pathway (Rajendran, L. et al. Proc. Natl. Acad. Sci. U.S.A 2006 103:11172-11177), and AR peptides are released as cargo inside extracellular vesicles capable of a neuron to neuron transmission of the toxic AR peptides (Sardar Sinha, M. et al. Acta Neuropathol. 2018 136:41-56). Extracellular Vesicles (EVs), recently emerged as key players in intercellular communication by the transport of functional cargo such as DNA, RNA, and proteins (Traci, N., et al. Int. J. Mol. Sci. 2016 17:171). Most cell types release EVs and in the skin EVs release has been characterized in the context of cell-cell communication, wound healing and cutaneous disorders (Terlecki-Zaniewicz, L. et al. J. Invest. Dermatol. 2019 139:2425-2436.e5). Disclosed herein is the characterization of a transcriptome of skin-derived EVs from the 3×Tg-AD triple transgenic mouse model of AD, confirming the presence of mRNA of APP and Tau and how these molecules can be transferred to neurons via cellular uptake.

Therefore, as disclosed herein, the skin can dispatch signals to the brain in the form of exosomes, and exosomes derived from the skin of Alzheimer's Disease (AD) subjects contain neurotoxic cargo that could potentially be impacting the progression of this disease. This is a paradigm-shifting concept in AD, referred to herein as a “skin-brain axis.” Disclosed herein are methods of using this phenomenon to diagnose neurological disease by assaying skin-derived exosomes from the subject, as well as methods of treating a subject with a neurological disease by modifying skin-derived exosomes with therapeutic cargo that will be delivered to the brain of the subject.

### Diagnostic

Therefore, disclosed herein is a method for diagnosing a neurodegenerative disease in a subject that involves isolating skin-derived exosomes from the subject and assaying the exosomes for the presence of one or more biomarkers. I

Therefore, disclosed herein is a method for diagnosing a neurodegenerative disease in a subject that involves isolated exosomes from the subject and assaying the exosomes for the presence of one or more biomarkers. In some embodiments, the biomarker is S100A8, S100A9, MAPK13, or a combination thereof. In some embodiments, the biomarker is APP (NC_000021.9), Aβ, or Tau, or a combination thereof.

In some embodiments, the biomarker is MAPT (NC_000017.11), ADAMTS9 (NC_000003.12), AKAP5 (NC_000014.9), AQP1 (NC_000007.14), ARC (NC_000008.11), CAMK2A (NC_000005.10), CASP4 (NC_000011.10), CLEC3B (NC_000003.12), FAAH (NC_000001.11), GRIN1 (NC_000009.12), HDAC9 (NC_000007.14), MAP2K3 (NC_000017.11), S100A8 (NC_000001.11), S100A9 (NC_000001.11), S100B (NC_000021.9), SLC16A6 (NC_000017.11), SLC1A3 (NC_000005.10), SLC25A4 (NC_000004.12), SLC30A3 (NC_000002.12), SLC38A2 (NC_000012.12), SLC39A3 (NC_000019.10), or any combination thereof.

In some embodiments, the biomarker is Rn45s, Mylpf, Neb, Rps3a1, Acta1, Atp2a1, Ckm, Ttn, Rnu11, Myh2, Xirp2, Lars2, Tnnc2, Dpt, Eno3, Elovl4, Des, Nr1d1, Tpm2, Tnnt3, Jph2, Smtnl1, Fbn1, Fmod, Serpina3j, Tnni2, Cmya5, Hspb6, Malat1, S100a9, Slc25a4, Trdn, Hfe2, Mybpc1, Casq1, Ryr1, Car3, Clec3b, Gas6, Fbxo40, Slc38a2, Myo18b, BC100530, Lor, Kdm6b, Myh6, Akr1e1, Cacna1s, Ogn, Spon2, Fh11, Icam1, Myoc, Rnase2b, Mb, Mxd1, Gbp2b, Dhcr24, Mybph, Igfbp6, Alpk3, Gltp, Paqr7, Krt6b, Nr1d2, Dkk2, Igfn1, Hydin, Gm13177, Mypn, Cdsn, Myl1, Casc1, Dbp, Clca2, Tcea3, Anxa3, Hrc, L2hgdh, Spp1, Arid5b, Tpm1, Pcf11, Scarna2, Col3a1, Pttg1, Asb2, Krt6a, Dsc3, Trim54, Baiap3, 2310002L09Rik, Tgfb2, Il1b, Cilp, Pfkm, Cdr1, Gm13483, Scn10a, Slc17a7, Myh4, Camk2a, Cxcl2, Pcio, Mt1, Tnfaip3, Cntn2, Klh131, Mstn, Dsp, Pcolce2, Fam207a, Tnnt1, Asic1, Tagap, Asb5, Fxyd2, Srl, Ldb3, Ryr3, Nrap, Mybpc2, Gse1, Abra, Ildr1, Smyd1, Hmox1, Obscn, Grin1, Stk36, S100a8, Zfp560, Fsd2, Polq, Thbs4, Gsdma, Krt80, 1-Mar, Fibin, Krt7, Pik3cg, Bcan, Flnc, Ndufv2, Tacstd2, Myoz1, Zxdb, Il11ra1, Neurl1a, Rbfox1, Cdh1, Fam83g, Snora23, Plet1, Zfp799, Taf15, Cstf1, 3425401B19Rik, Larp7, Slfn4, Gm13375, Ppp1r3a, Mmp15, A1cf, Chst3, F630111L10Rik, Gbp11, Gm6583, Lrguk, Mcidas, Rnf182, Syt2, Tsks, Ugt1a1, Mgme1, Dnah8, Dock10, Lama2, Arntl, Mitf, Fuca2, Kif9, Myl2, Adamts9, Jak2, Fndc5, S100b, Ces1d, Pygm, Dnah5, Ncan, Ppp1r3f, Hcar2, Nov, Ugcg, Arc, Tcf711, Tmem182, Ddhd1, A130010J15Rik, Hps3, Rlim, Hdac9, Naicn, Fbxo17, Helb, Cd93, Cyp2b23, Gdpd3, Slfn10-ps, Trim72, Abat, Nup37, Zc3h12c, Kat2b, Agpat6, Vma21, Syn1, Pitrm1, Igfbp2, Pvaib, Nfkbiz, Hist1h2ab, Actn3, Myo5b, Dsc1, Sowahc, Sptbn2, Dna2, Eomes, Otog, Sike1, Ush1g, Muc19, Mroh4, Rbm46, Rimbp3, Gcnt2, Myh7, Cxcl14, Tep1, Myom2, Stfa3, Chil3, Sema7a, Vsx2, Nfkb1, Ovol1, Acss1, Zfp719, Prr12, Myom3, Arrdc4, H19, Dmpk, Sik1, Elovl6, Gspt2, Radii, Mfng, Atp6v1b2, Etv3, Rnasek, Schip1, Epha2, 2310003H01Rik, Saa3, Mfap4, Slc38a3, Stfa1, Mmp25, Zgrf1, Epha4, Mdm2, Lphn3, Arid2, Map2k3, Tgfbr1, Ckap2, Rilpl1, Srgn, Duox1, Cadps2, Wdr8, Vav2, Gna15, Tef, Ifit3, Limch1, Mmp17, Rasgrp3, B3glct, Pnpla1, Sfi1, Mgl2, Rad9a, Rap1gap, Utp23, Tbl1x, Wdr60, Casp4, Rab11fip1, Rbm20, Ptpn9, Snora64, Nrip1, Glp1r, 9530051G07Rik, Neurod1, Sh3gl3, Tex16, BC068157, Dennd6b, Setmar, Gtf2ird2, Shank3, Gm7120, Atp8b3, Shisa4, Sqle, Klh141, Tmem198, 1810013L24Rik, Slc1a3, Tsc22d2, Emp2, F3, Hmga2-ps1, Xirp1, Rabl2, Speg, Mon1a, Dtna, Agap3, Phactr4, Piezo1, Spta1, Foxp2, Atp2b4, Crtc2, Gpr34, Lama5, Cdh5, Hrnr, Islr2, Plk2, Celsr1, Snord15a, Nhlrc1, St3gal3, Mapk13, Scarna13, Ccl4, Spint2, Jpx, Ankrd26, Efna1, Ggct, H2-T23, Myot, Pcca, Cacna2d2, Kpna7, Msh5, Batf, Lgals4, Fbxo30, Synpo2, 4930563E22Rik, C730027H18Rik, Fbxo27, Foxa2, Lrriq1, Npy6r, Pld5, Pth2r, Rab3c, Scgb3a2, Slc17a3, Slc6a7, Spata31d1d, Srrm4os, Tigd4, Tmco5, Vwa7, Gpr56, Adssl1, Pard6b, Usp53, Itch, Col24a1, Vipr1, Gm13178, Lrrc39, Cntnap1, Myh3, Slfn9, Slc16a6, 4930431P03Rik, 9130204L05Rik, Ghsr, Icos, Slc26a3, Trappc3l, Unc5d, Pim1, Agtr1a, Tie1, Dancr, Phkb, Ubl3, Irf1, Smtn, PIk3, Efnb2, Abca8b, Lgals6, Tmppe, Pde4d, Clec2i, Gba, Brwd1, Smpx, Sprr2h, Fermt1, Chd3os, 5-Mar, Pkia, Spock2, Rhebl1, Col22a1, Amot, Pde4b, 9030624J02Rik, Pecam1, Gsto1, Gadl1, Lep, Gabra3, Kdm2b, Fndc9, Alpk2, Ptgs2, Tob2, Ccdc61, Fgf10, Scn4a, AU018091, Sumf1, Mir6516, Abca12, Sdr16c5, Txlnb, Snord22, Palm3, Itsn2, Pmm1, Coq10b, Aga, Selp, Mfap5, Slc16a13, Gtf2h3, Zswim4, Greb1, Gna12, Ddit4l, Cuedc2, Mrpl44, Gm15800, Ldlrad1, Misp, Nphp4, Cd14, Pfkfb1, Slc41a3, Herc4, Cadm4, Scarf2, Ckmt2, C030039L03Rik, Fkbp5, Wnk2, Hmces, Zbtb9, Hoxc10, Ccnl1, Cldn4, Ccl3, Eef2k, Irg1, Cpne7, Aqp1, A930013F10Rik, Exo1, Csf3, Dsc2, Trpm5, Fbxw17, Tmem150b, Ggps1, Bhlhb9, Hs1bp3, Fam102a, Pcyox1l, Fnip2, Zfp637, Cpt1b, Il31ra, C920009B18Rik, Klk7, Sp140, Rnf122, Adamts17, Irf6, Podx1, Klb, Dusp1, Dus1l, Slc2a4, Pgr, Snora26, D430019H16Rik, Fbxl15, Adamtsl4, Sbf1, Ccdc71l, Slc39a3, Col4a4, Ras110b, Sspo, Tmf1, C030006K11Rik, Fundc2, Hccs, Olfml2a, Nppb, Plaur, Pld3, Hhipl1, Dusp13, Gpr171, Igf2bp3, Sfxn4, Adss, Tollip, Lmtk2, Sele, Nup62, Dusp5, Csrp3, Akap5, Myo1a, Onecut2, Clec11a, Ssc5d, Faah, Per2, Rfxap, Zfp131, Ippk, Endov, Top3b, Szt2, Zbtb16, Aacs, BC024139, Stx11, Stambpl1, Pcdh1, Trim45, Ttll8, Il2rg, Serpina3h, Sptbn4, Alox8, Lmod3, Arf2, Fosl1, Yae1d1, H2-Q4, Slc16a12, Stoml1, Epdr1, Bcl9l, Bpifc, Nuak1, Primpol, Abcb1b, Abca17, Abcc6, Cfi, Slc30a3, Tmem151b, Mylk2, Pde12, Lsr, C530008M17Rik, Nap1l5, Nos2, P4ha3, E330033B04Rik, Myo5c, 4931406P16Rik, Ugt1a7c, Dedd2, Cacna1b, 2810417H13Rik, Acvr2b, Hbegf, Fbxw9, Mfap3, A1661453, Fdx1, Ankrd55, Imp3, Grhl3, Map3k11, Hdc, Dennd3, Gpd1l, Akap1, R3hcc1, Procr, Adhfe1, 3-Sep, AY512915, Catsperg2, Cngb3, Dnaaf1, Fam132b, Fbxw18, Fuom, Gm5878, Oprl1, Slc6a13, Eef1a2, Alkbh1, Ccdc175, Clec4g, Kazald1, Ttbk1, Snora44, Acot6, Ralgds, Naa35, Gipcl, Ugt1a2, Ugt1a5, Ugt1a9, Fcer2a, Gp49a, Fiz1, Angptl7, Anks1b, Edn2, Fank1, Fgf6, Gm13032, Kbtbd8, Kcnq2, Mrgprx2, Fv1, Rab1b, Rasip1, Alox12b, or any combination thereof, including any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, 93, 94, 95, 96, 97, 98, 99, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 420, 430, 440, 441, 442, 443, 444, 445, 456, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 661, 662, 664, 665, or 666 of these biomarkers.

In some embodiments, the biomarker is a gene involved in Alzheimer's Disease, such as Abat, Adamts9, Adss, Akap5, Alox12b, Ankrd55, Aqp1, Arc, Asb2, Atp6v1b2, Batf, Cadps2, Camk2a, Casp4, Ccl3, Ccl4, Cd14, Cd93, Ces1d, Cfi, Chst3, Clec3b, Cpne7, Cpt1b, Crtc2, Cxcl14, Dhcr24, Dsc2, Dtna, Dusp1, Eef2k, Elovl6, Epha2, Epha4, F3, Faah, Fbxo27, Fbxw9, Fkbp5, Fndc5, Fndc9, Gabra3, Gba, Gcnt2, Ggct, Ggps1, Grin1, Gsto1, Hdac9, Hmox1, Icam1, Igfbp2, Igfbp6, Il1b, Il31ra, Irf1, Itsn2, Kif9, Lama5, Ldb3, Lep, Lor, Lrrc39, Malat1, Map2k3, Mfap4, Mfng, Mybpc2, Myo5b, Myot, Nalcn, Nap1l5, Nfkb1, Nfkbiz, Nhlrc1, Nos2, Ogn, Pc/o, Pcyox1l, Pecam1, Per2, Pfkm, Pgr, Piezo1, Pik3cg, Pim1, Plaur, Pld3, Plk2, Pmm1, Ptgs2, Rab3c, Rbfox1, Rhebl1, Rnase2b, S100a8, S100a9, S100b, Se/p, Slc16a6, Slc1a3, Slc25a4, Slc30a3, Slc38a2, Slc39a3, Smtn, Sowahc, Spon2, Spp1, St3gal3, Stk36, Stoml1, Stx11, Tacstd2, Tbl1x, Tcf7l1, Tep1, Tgfb2, Tgfbr1, Tmem198, Tnfaip3, Tob2, Trim45, Trim54, Unc5d, Vipr1, Zbtb16, Zc3h12c, or any combination thereof.

In some embodiments, the method involves assaying the exosomes for the regulation of one or more pathways related to neurodegenerative disease. For example, the method can involve assaying for calcium signaling, HMGB1 signaling, IL-6 signaling, and IL-8 signaling. In some embodiments, the method involves assaying the exosomes for the regulation of one or more pathways related to immune and inflammatory responses. For example, the method can involve assaying accumulation of neutrophils, migration of myeloid cells, activities of IL-6 and IL-8, or any combination thereof.

For example, the method can involve assaying for dysregulation of any pathway associated with a brain disorder, disease, or injury. For example, the pathway can be a Neuroinflammation Signaling Pathway, GP6 Signaling Pathway, Natural Killer Cell Signaling, HMGB1 Signaling, IL-6 Signaling, Actin Cytoskeleton Signaling, ILK Signaling, Factors Promoting Cardiogenesis in Vertebrates, IL-8 Signaling, Xenobiotic Metabolism AHR Signaling Pathway, IL-15 Production, Hepatic Fibrosis Signaling Pathway, Xenobiotic Metabolism General Signaling Pathway, Osteoarthritis Pathway, LXR/RXR Activation, Type I Diabetes Mellitus Signaling, Netrin Signaling, Dendritic Cell Maturation, Thyroid Hormone Metabolism II (via Conjugation and/or Degradation), Melatonin Degradation I, Nicotine Degradation II, Superpathway of Melatonin Degradation, Antioxidant Action of Vitamin C, TREM1 Signaling, Nicotine Degradation III, Cardiac Hypertrophy Signaling (Enhanced), Protein Kinase A Signaling, HIF1α Signaling, Mouse Embryonic Stem Cell Pluripotency, Gαi Signaling, Retinol Biosynthesis, Insulin Secretion Signaling Pathway, Calcium Signaling, HOTAIR Regulatory Pathway, Endothelin-1 Signaling, Xenobiotic Metabolism PXR Signaling Pathway, Semaphorin Neuronal Repulsive Signaling Pathway, Pancreatic Adenocarcinoma Signaling, Serotonin Degradation, Acute Phase Response Signaling, Toll-like Receptor Signaling, iNOS Signaling, Synaptogenesis Signaling Pathway, IL-17A Signaling in Airway Cells, p38 MAPK Signaling, Cardiac Hypertrophy Signaling, STAT3 Pathway, Th2 Pathway, Endocannabinoid Neuronal Synapse Pathway, Leukocyte Extravasation Signaling, Intrinsic Prothrombin Activation Pathway, Gα12/13 Signaling, PEDF Signaling, MIF Regulation of Innate Immunity, IL-23 Signaling Pathway, Paxillin Signaling, ErbB Signaling, Crosstalk between Dendritic Cells and Natural Killer Cells, Xenobiotic Metabolism CAR Signaling Pathway, Phospholipase C Signaling, CD40 Signaling, PKC Signaling in T Lymphocytes, AMPK Signaling, Regulation Of The Epithelial Mesenchymal Transition By Growth Factors Pathway, RhoA Signaling, GNRH Signaling, Colorectal Cancer Metastasis Signaling, Acetone Degradation I (to Methylglyoxal), Xanthine and Xanthosine Salvage, Glutathione-mediated Detoxification, Guanine and Guanosine Salvage I, Docosahexaenoic Acid (DHA) Signaling, Adenine and Adenosine Salvage I, Histamine Biosynthesis, Acetate Conversion to Acetyl-CoA, Role of IL-17A in Arthritis, VDR/RXR Activation, Tight Junction Signaling, Glucocorticoid Receptor Signaling, Thyroid Hormone Biosynthesis, Aldosterone Signaling in Epithelial Cells, Superpathway of Cholesterol Biosynthesis, MSP-RON Signaling Pathway, Adenine and Adenosine Salvage III, Purine Nucleotides De Novo Biosynthesis II, Bladder Cancer Signaling, Antigen Presentation Pathway, Phospholipases, Arsenate Detoxification I (Glutaredoxin), Creatine-phosphate Biosynthesis, Cholesterol Biosynthesis I, Methylglyoxal Degradation III, Systemic Lupus Erythematosus Signaling, IL-10 Signaling, Th1 and Th2 Activation Pathway, Ephrin A Signaling, Prostanoid Biosynthesis, Role of Hypercytokinemia/hyperchemokinemia in the Pathogenesis of Influenza, Calcium Transport I, IL-17 Signaling, Xenobiotic Metabolism Signaling, Eicosanoid Signaling, Iron homeostasis signaling pathway, Atherosclerosis Signaling, Triacylglycerol Degradation, Oncostatin M Signaling, Differential Regulation of Cytokine Production in Macrophages and T Helper Cells by IL-17A and IL-17F, Geranylgeranyldiphosphate Biosynthesis, Circadian Rhythm Signaling, Adenosine Nucleotides Degradation II, Estrogen-mediated S-phase Entry, The Visual Cycle, Apelin Cardiac Fibroblast Signaling Pathway, cAMP-mediated signaling, Communication between Innate and Adaptive Immune Cells, Purine Ribonucleosides Degradation to Ribose-1-phosphate, GABA Receptor Signaling, Cholesterol Biosynthesis III (via Desmosterol), Histidine Degradation VI, Epithelial Adherens Junction Signaling, Granulocyte Adhesion and Diapedesis, Role of IL-17A in Psoriasis, Zymosterol Biosynthesis, Choline Biosynthesis III, Heme Degradation, Bupropion Degradation, Purine Nucleotides Degradation II (Aerobic), Agranulocyte Adhesion and Diapedesis, Epoxysqualene Biosynthesis, Phagosome Maturation, Differential Regulation of Cytokine Production in Intestinal Epithelial Cells by IL-17A and IL-17F, Trans, trans-farnesyl Diphosphate Biosynthesis, Role of Tissue Factor in Cancer, MIF-mediated Glucocorticoid Regulation, Apelin Adipocyte Signaling Pathway, BAG2 Signaling Pathway, DNA damage-induced 14-3-3a Signaling, Regulation of the Epithelial-Mesenchymal Transition Pathway, L-carnitine Biosynthesis, Estrogen Biosynthesis, RAR Activation, Cholesterol Biosynthesis II (via 24,25-dihydrolanosterol), Germ Cell-Sertoli Cell Junction Signaling, Hepatic Fibrosis/Hepatic Stellate Cell Activation, 4-aminobutyrate Degradation I, eNOS Signaling, Retinoate Biosynthesis I, IL-7 Signaling Pathway, Fatty Acid α-oxidation, Role of IL-17F in Allergic Inflammatory Airway Diseases, Cellular Effects of Sildenafil (Viagra), Graft-versus-Host Disease Signaling, nNOS Signaling in Skeletal Muscle Cells, Anandamide Degradation, Apelin Liver Signaling Pathway, Vitamin-C Transport, or any combination thereof.

In some aspects, the method is an immunoassay. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

In some aspects, the method comprises detecting mRNA biomarkers. A number of widely used procedures exist for detecting and determining the abundance of a particular mRNA in a total or poly(A) RNA sample. For example, specific mRNAs can be detected using Northern blot analysis, nuclease protection assays (NPA), in situ hybridization, or reverse transcription-polymerase chain reaction (RT-PCR). In some embodiments, the method involves qRT-PCR, digital PCR, or in situ hybridization with molecular beacons or molecular flares/probes.

### Modified Skin-Derived Extracellular Vehicles (EVs)

Disclosed herein are methods of treating a subject with a neurological disease by modifying skin-derived exosomes with therapeutic cargo that will be delivered to the brain of the subject. In some embodiments, the method involves engineering the skin of the subject to produce therapeutic exosomes. In some embodiments, the method involves collecting skin-produced exosomes and loading them with therapeutic cargo.

Engineering Skin Cells to Produce Therapeutic EVs

Also disclosed are methods of reprogramming skin cells into EV-producing cells that involve delivering intracellularly into the skin cells a polynucleotide comprising nucleic acid sequences encoding therapeutic genes.

In some embodiments, this method involves transfecting the skin of the subject with an expression vector encoding an anti-Tau siRNA, miRNA, or any combination thereof. In some embodiments, this method involves transfecting the skin of the subject with an expression vector encoding one or more anti-inflammatory genes, such as siRNAs, or mRNAs that reduce glial cell activity.

In some embodiments, this method involves transfecting the skin of the subject with an expression vector encoding one or more vasculogenic factors, such as Etv2 (NM_001300974.2, NM_001304549.2, NM_014209.4), Foxc2 (NM_005251.3), Fli1 (NM_001167681.2, NM_001271010.1, NM_001271012.1, NM_002017.5), VEGFA (NM_001025366.3, NM_001025367.3, NM_001025368.3, NM_001025369.3, NM_001025370.3 NM_001033756.3, NM_001171622.2, NM_001171623.1, NM_001171624.1, NM_001171625.1, NM_001171626.1, NM_001171627.1, NM_001171628.1, NM_001171629.1, NM_001171630.1, NM_001204384.1, NM_001204385.2, NM_001287044.2, NM_001317010.1, NM_003376.6), VEGFB (NM_003377.5, NM_001243733.2), VEGFC (NM_005429.5), VEGFD (NM_004469.5), bFGF (NM_001361665.2, NM_002006.5), Sox17 (NM_022454.4), Oct4, Klf4 (NM_001314052.2, NM_004235.6), or any combination thereof.

In some embodiments, this method involves transfecting the skin of the subject with an expression vector encoding one or more neurogenic factors, such as Ascl1 (NM_004316.4), Ascl2 (NM_005170.3), Ascl3 (NM_020646.2), Ascl5 (NM_001270601.1), Neurog1 (NM_006161.3), Neurog2 (NM_024019.4), Neurog3 (NM_020999.4), Neurod1 (NM_002500.5), Neurod2 (NM_006160.4), Neurod4 (NM_021191.3), Neurod6 (NM_022728.4), Atoh1 (NM_005172.2), Atoh7 (NM_145178.4), Atoh8 (NM_032827.7), Myf5 (NM_005593.3), Ptf1a (NM_178161.3), Brn3c (NM_002700.3), Brn3a (NM_006237.4), Brn3b (NM_004575.3), Brn1 (NM_006236.3), Brn2 (NM_005604.4), Brn4 (NM_000307.5), Oct4 (NM_001173531.2), Oct6 (NM_002699.4), Pit1 (NM_000306.4), Brn5 (NM_001330422.2), Myt1I (NM_001303052.2), Nurr1 (NM_006186.4), or any combination thereof.

In some embodiments, this method involves transfecting the skin of the subject with an expression vector encoding an anti-APP siRNA, miRNA, such as hsa-miR-106b-5p, hsa-miR-101-3p, hsa-miR-520c-3p, hsa-miR-106a-5p, hsa-miR-20a-5p, hsa-miR-17-5p, hsa-miR-15a-5p, hsa-miR-130a-3p, hsa-let-7d-5p, hsa-let-7a-5p, hsa-miR-16-5p, hsa-miR-144-3p, hsa-miR-4422, hsa-let-7f-1-3p, hsa-let-7a-3p, hsa-let-7b-3p, hsa-miR-98-3p, hsa-miR-380-3p, hsa-miR-6835-3p, hsa-miR-4772-5p, hsa-miR-101-3p, hsa-miR-4719, hsa-miR-520f-3p, hsa-miR-3908, hsa-miR-4269, hsa-miR-323a-3p, hsa-miR-6715b-5p, hsa-miR-153-3p, hsa-miR-4495, hsa-miR-4786-5p, hsa-miR-3911, hsa-miR-6085, hsa-miR-6813-5p, or any combination thereof.

In some embodiments, this method involves transfecting the skin of the subject with an expression vector encoding an anti-MAPT siRNA, miRNA, such as hsa-miR-34c-5p, hsa-miR-657, hsa-miR-4728-5p, hsa-miR-3978, or any combination thereof.

In some embodiments, this method involves transfecting the skin of the subject with an expression vector encoding an anti-Inflammatory gene, such as PPARγ (NM_001330615.4, NM_001354666.3, NM_001354667.3, NM_001354668.2, NM_001354669.2, NM_001354670.2, NM_001374261.3, NM_001374262.3, NM_001374263.2, NM_001374264.2, NM_001374265.1, NM_001374266.1, NM_005037.7, NM_015869.5, NM_138711.6, NM_138712.5), Il-10 (NM_000572.3, NM_001382624.1), Il-27 (NM_145659.3), Trem2 (NM_001271821.2, NM_018965.4), IkBα (NM_020529.3), Sirt1 (NM_001142498.1, NM_001314049.1, NM_012238.5), or any combination thereof.

In some embodiments, this method involves transfecting the skin of the subject with an expression vector encoding an siRNA or miRNA targeting a pro-inflammatory gene. In some embodiments, the miRNA can be an anti-P2X₄R miRNA, such as hsa-miR-335-5p, hsa-miR-106b-5p, or hsa-miR-20a-5p. In some embodiments, the miRNA can be an anti-TLR4 miRNA, such as hsa-let-7i-5p, hsa-miR-146a-5p, hsa-miR-335-5p, hsa-miR-146b-5p, hsa-let-7b-5p, hsa-miR-448, or hsa-miR-3924. In some embodiments, the miRNA can be an anti-CX3CR1 miRNA, such as hsa-miR-296-3p, hsa-miR-1227-3p, hsa-miR-4261, or hsa-miR-147b-5p. In some embodiments, the miRNA can be an anti-IL-1β miRNA, such as hsa-miR-204-5p, hsa-miR-21-5p, hsa-miR-887-3p, hsa-miR-24-3p, hsa-miR-106a-5p, hsa-miR-877-3p, hsa-miR-5692a, hsa-miR-5688, or hsa-miR-495-3p.

In some embodiments, the nucleic acid sequences are present in non-viral vectors. In some embodiments, the nucleic acid sequences are operably linked to an expression control sequence. In other embodiments the nucleic acids are operably linked to two or more expression control sequences.

A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

In some embodiments, after transfecting target cells, the cells can then pack the transfected genes (e.g. cDNA, miRNA, etc. . . . ) into EVs, which can then induce other skin cells to form EV-producing cells. Therefore, also disclosed is a method of reprogramming skin cells into EV-producing cells that involves exposing the somatic cell with an extracellular vesicle produced from a cell containing or expressing the disclosed therapeutic genes.

Therefore, disclosed are methods of reprogramming skin cells into EV-producing cells that involve exposing the skin cells to extracellular vesicles (EVs) isolated from cells expressing or containing exogenous polynucleotides comprising one or more nucleic acid sequences encoding the disclosed therapeutic genes. EVs secreted by the donor cells can then collected from the culture medium. These EVs can then be administered to the skin cells to reprogram them into insulin-producing cells. In some embodiments, the donor cells can be any cell from the subject able to produce EVs, including (but not limited to) skin cells (e.g., fibroblasts, keratinocytes, skin stem cells), adipocytes, dendritic cells, peripheral blood mononuclear cells (PBMC), pancreatic cells (e.g., ductal epithelial cells), liver cells (e.g., hepatocytes), immune cells (e.g., T cells, macrophages, myeloid derived suppressor cells).

Disclosed herein are compositions and methods for reprogramming skin cells into EV-producing cells both in vitro and in vivo that can be used to treat neurological diseases.

Exosomes and microvesicles are EVs that differ based on their process of biogenesis and biophysical properties, including size and surface protein markers. Exosomes are homogenous small particles ranging from 40 to 150 nm in size and they are normally derived from the endocytic recycling pathway. In endocytosis, endocytic vesicles form at the plasma membrane and fuse to form early endosomes. These mature and become late endosomes where intraluminal vesicles bud off into an intra-vesicular lumen. Instead of fusing with the lysosome, these multivesicular bodies directly fuse with the plasma membrane and release exosomes into the extracellular space. Exosome biogenesis, protein cargo sorting, and release involve the endosomal sorting complex required for transport (ESCRT complex) and other associated proteins such as Alix and Tsg101. In contrast, microvesicles, are produced directly through the outward budding and fission of membrane vesicles from the plasma membrane, and hence, their surface markers are largely dependent on the composition of the membrane of origin. Further, they tend to constitute a larger and more heterogeneous population of extracellular vesicles, ranging from 150 to 1000 nm in diameter. However, both types of vesicles have been shown to deliver functional mRNA, miRNA and proteins to recipient cells.

In some embodiments, the polynucleotides are delivered to the somatic cells, or the donor cells for EVs, intracellularly via a gene gun, a microparticle or nanoparticle suitable for such delivery, transfection by electroporation, three-dimensional nanochannel electroporation, a tissue nanotransfection device, a liposome suitable for such delivery, or a deep-topical tissue nanoelectroinjection device. In some embodiments, a viral vector can be used. However, in other embodiments, the polynucleotides are not delivered virally.

Electroporation is a technique in which an electrical field is applied to cells in order to increase permeability of the cell membrane, allowing cargo (e.g., reprogramming factors) to be introduced into cells. Electroporation is a common technique for introducing foreign DNA into cells.

Tissue nanotransfection allows for direct cytosolic delivery of cargo (e.g., reprogramming factors) into cells by applying a highly intense and focused electric field through arrayed nanochannels, which benignly nanoporates the juxtaposing tissue cell members, and electrophoretically drives cargo into the cells.

In order to express a polypeptide or functional nucleic acid, the nucleotide coding sequence may be inserted into appropriate expression vector. Therefore, also disclosed is a non-viral vector comprising a polynucleotide comprising nucleic acid sequences disclosed herein, wherein the nucleic acid sequences are operably linked to an expression control sequence. In some embodiments, the nucleic acid sequences are operably linked to a single expression control sequence. In other embodiments, the nucleic acid sequences are operably linked to two or more separate expression control sequences.

Methods to construct expression vectors containing genetic sequences and appropriate transcriptional and translational control elements are well known in the art. These methods include in vitro recombinant DNA techniques, synthetic techniques, and in vivo genetic recombination. Such techniques are described in Sambrook et al., Molecular Cloning, A Laboratory Manual (Cold Spring Harbor Press, Plainview, N.Y., 1989), and Ausubel et al., Current Protocols in Molecular Biology (John Wiley & Sons, New York, N.Y., 1989).

Expression vectors generally contain regulatory sequences necessary elements for the translation and/or transcription of the inserted coding sequence. For example, the coding sequence is preferably operably linked to a promoter and/or enhancer to help control the expression of the desired gene product.

Promoters used in biotechnology are of different types according to the intended type of control of gene expression. They can be generally divided into constitutive promoters, tissue-specific or development-stage-specific promoters, inducible promoters, and synthetic promoters.

Constitutive promoters direct expression in virtually all tissues and are largely, if not entirely, independent of environmental and developmental factors. As their expression is normally not conditioned by endogenous factors, constitutive promoters are usually active across species and even across kingdoms. Examples of constitutive promoters include CMV, EF1a, SV40, PGK1, Ubc, Human beta actin, and CAG.

Tissue-specific or development-stage-specific promoters direct the expression of a gene in specific tissue(s) or at certain stages of development. For plants, promoter elements that are expressed or affect the expression of genes in the vascular system, photosynthetic tissues, tubers, roots and other vegetative organs, or seeds and other reproductive organs can be found in heterologous systems (e.g. distantly related species or even other kingdoms) but the most specificity is generally achieved with homologous promoters (i.e. from the same species, genus or family). This is probably because the coordinate expression of transcription factors is necessary for regulation of the promoter's activity.

The performance of inducible promoters is not conditioned to endogenous factors but to environmental conditions and external stimuli that can be artificially controlled. Within this group, there are promoters modulated by abiotic factors such as light, oxygen levels, heat, cold and wounding. Since some of these factors are difficult to control outside an experimental setting, promoters that respond to chemical compounds, not found naturally in the organism of interest, are of particular interest. Along those lines, promoters that respond to antibiotics, copper, alcohol, steroids, and herbicides, among other compounds, have been adapted and refined to allow the induction of gene activity at will and independently of other biotic or abiotic factors.

The two most commonly used inducible expression systems for research of eukaryote cell biology are named Tet-Off and Tet-On. The Tet-Off system makes use of the tetracycline transactivator (tTA) protein, which is created by fusing one protein, TetR (tetracycline repressor), found in Escherichia coli bacteria, with the activation domain of another protein, VP16, found in the Herpes Simplex Virus. The resulting tTA protein is able to bind to DNA at specific TetO operator sequences. In most Tet-Off systems, several repeats of such TetO sequences are placed upstream of a minimal promoter such as the CMV promoter. The entirety of several TetO sequences with a minimal promoter is called a tetracycline response element (TRE), because it responds to binding of the tetracycline transactivator protein tTA by increased expression of the gene or genes downstream of its promoter. In a Tet-Off system, expression of TRE-controlled genes can be repressed by tetracycline and its derivatives. They bind tTA and render it incapable of binding to TRE sequences, thereby preventing transactivation of TRE-controlled genes. A Tet-On system works similarly, but in the opposite fashion. While in a Tet-Off system, tTA is capable of binding the operator only if not bound to tetracycline or one of its derivatives, such as doxycycline, in a Tet-On system, the rtTA protein is capable of binding the operator only if bound by a tetracycline. Thus the introduction of doxycycline to the system initiates the transcription of the genetic product. The Tet-On system is sometimes preferred over Tet-Off for its faster responsiveness.

In some embodiments, the nucleic acid sequences disclosed herein are operably linked to the same expression control sequence. Alternatively, internal ribosome entry sites (IRES) elements can be used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Disclosed are non-viral vectors containing one or more polynucleotides disclosed herein operably linked to an expression control sequence. Examples of such non-viral vectors include the oligonucleotide alone or in combination with a suitable protein, polysaccharide or lipid formulation. Non-viral methods present certain advantages over viral methods, with simple large scale production and low host immunogenicity being just two. Previously, low levels of transfection and expression of the gene held non-viral methods at a disadvantage; however, recent advances in vector technology have yielded molecules and techniques with transfection efficiencies similar to those of viruses.

Examples of suitable non-viral vectors include, but are not limited to pIRES-hrGFP-2a, pCMV6, pMAX, pCAG, pAd-IRES-GFP, and pCDNA3.0.

The compositions disclosed can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Modified Skin-Derived EVs

Also disclosed are methods of collecting skin-produced exosomes and loading them with therapeutic cargo. The disclosed EVs can in some embodiments be any vesicle that can be secreted by a cell. Cells secrete extracellular vesicles (EVs) with a broad range of diameters and functions, including apoptotic bodies (1-5 μm), microvesicles (100-1000 nm in size), and vesicles of endosomal origin, known as exosomes (50-150 nm).

The disclosed extracellular vesicles may be prepared by methods known in the art. For example, the disclosed extracellular vesicles may be prepared by expressing in a eukaryotic cell an mRNA that encodes the cell-targeting ligand. In some embodiments, the cell also expresses an mRNA that encodes a therapeutic cargo. The mRNA for the cell-targeting ligand and the therapeutic cargo may be expressed from vectors that are transfected into suitable production cells for producing the disclosed EVs. The mRNA for the cell-targeting ligand and the therapeutic cargo may be expressed from the same vector (e.g., where the vector expresses the mRNA for the cell-targeting ligand and the therapeutic cargo from separate promoters), or the mRNA for the cell-targeting ligand and the therapeutic cargo may be expressed from separate vectors. The vector or vectors for expressing the mRNA for the cell-targeting ligand and the therapeutic cargo may be packaged in a kit designed for preparing the disclosed extracellular vesicles.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

### Therapeutic Cargo

The disclosed extracellular vesicles may be loaded with a therapeutic agent, where the extracellular vesicles deliver the agent to a target cell. Suitable therapeutic agents include but are not limited to therapeutic drugs (e.g., small molecule drugs), therapeutic proteins, and therapeutic nucleic acids (e.g., therapeutic RNA). In some embodiments, the disclosed extracellular vesicles comprise a therapeutic RNA (also referred to herein as a “cargo RNA”).

For example, in some embodiments the fusion protein containing the cell-targeting motif also includes an RNA-domain (e.g., at a cytosolic C-terminus of the fusion protein) that binds to one or more RNA-motifs present in the cargo RNA in order to package the cargo RNA into the extracellular vesicle, prior to the extracellular vesicles being secreted from a cell. As such, the fusion protein may function as both of a “cell-targeting protein” and a “packaging protein.” In some embodiments, the packaging protein may be referred to as extracellular vesicle-loading protein or “EV-loading protein.”

In some embodiments, the cargo RNA is an miRNA, shRNA, mRNA, ncRNA, sgRNA or any combination thereof. For example, in some embodiments, the anti-inflammatory agent is micro-RNA 146a. Other miRNAs have been reported to regulate the expression of key molecules responsible for M1-favoring glycolytic metabolism (e.g., mRr9, miR127 and miR155).

The cargo RNA of the disclosed extracellular vesicles may be of any suitable length. For example, in some embodiments the cargo RNA may have a nucleotide length of at least about 10 nt, 20 nt, 30 nt, 40 nt, 50 nt, 100 nt, 200 nt, 500 nt, 1000 nt, 2000 nt, 5000 nt, or longer. In other embodiments, the cargo RNA may have a nucleotide length of no more than about 5000 nt, 2000 nt, 1000 nt, 500 nt, 200 nt, 100 nt, 50 nt, 40 nt, 30 nt, 20 nt, or 10 nt. In even further embodiments, the cargo RNA may have a nucleotide length within a range of these contemplated nucleotide lengths, for example, a nucleotide length between a range of about 10 nt-5000 nt, or other ranges. The cargo RNA of the disclosed extracellular vesicles may be relatively long, for example, where the cargo RNA comprises an mRNA or another relatively long RNA.

In some embodiments, the therapeutic cargo is a membrane-permeable pharmacological compound that is loaded into the EV after it is secreted by the cell. In some embodiments, the cargo is an anti-cancer agent that can cause apoptosis or pyroptosis of a targeted tumor cell. In some embodiments, the anti-cancer agent is a small molecule drug. For example, in some embodiments, the cargo is Ibrutinib. Additional examples of anti-cancer drugs or antineoplastics to be attached to the tumor targeting peptides described herein include, but are not limited to, aclarubicin, altretamine, aminopterin, amrubicin, azacitidine, azathioprine, belotecan, busulfan, camptothecin, capecitabine, carboplatin, carmofur, carmustine, chlorambucil, cisplatin, cladribine, clofarabine, cyclophosphamide, cytarabine, daunorubicin, decitabine, doxorubicin, epirubicin, etoposide, floxuridine, fludarabine, 5-fluorouracil, fluorouracil, gemcitabine, idarubicin, ifosfamide, irinotecan, mechlorethamine, melphalan, mercaptopurine, methotrexate, mitoxantrone, nedaplatin, oxaliplatin, paclitaxel, pemetrexed, pentostatin, pirarubicin, pixantrone, procarbazine, pyrimethamine raltitrexed, rubitecan, satraplatin, streptozocin, thioguanine, triplatin tetranitrate, teniposide, topotecan, tegafur, trimethoprim, uramustine, valrubicin, vinblastine, vincristine, vindesine, vinflunine, vinorelbine, and zorubicin.

To achieve loading of small RNAs into EVs, transfection-based approaches have been proposed. Other reports have shown that using vector-induced expression of small RNAs in cells, small RNA loading into EVs can be achieved. Alternatively, EV donor cells may be transfected with small RNAs directly. Incubation of tumor cells with chemotherapeutic drugs is also another method to package drugs into EVs. To stimulate formation of drug-loaded EVs, cells are irradiated with ultraviolet light to induce apoptosis. Alternative approaches such as fusogenic liposomes also leads loading drugs into EVs.

In some embodiments, the therapeutic cargo is loaded into the EVs by diffusion via a concentration gradient.

### Methods

Also contemplated herein are methods for using the disclosed EVs to treat a neurological disease. For example, the disclosed extracellular vesicles may be used for any injury, disease, or disorder of the brain by delivering a therapeutic gene or cargo. In some embodiments, the disclosed extracellular vesicles may be used to treat Spinal Cord Injury, Alzheimer's Disease, Amyotrophic Lateral Sclerosis, Ataxia, Cerebellar or Spinocerebellar Degeneration, Brain and Spinal Tumors, Cerebral Aneurysms, Epilepsy, Traumatic Brain Injury, Multiple Sclerosis, Parkinson's Disease, Stroke, Huntington's Disease, Autism Spectrum Disorder, Cerebral Palsy, Chronic Pain, Dementia With Lewy Bodies, Migraine, Niemann-Pick Disease, Frontotemporal Dementia, CADASIL, Spinocerebellar Degeneration and Atrophy, or any combination thereof.

The disclosed EVs may be administered to a subject by any suitable means. Administration to a human or animal subject may be selected from parenteral, intramuscular, intracerebral, intravascular, subcutaneous, or transdermal administration. Typically the method of delivery is by injection. Preferably the injection is intramuscular or intravascular (e.g. intravenous). A physician will be able to determine the required route of administration for each particular patient.

The EVs are preferably delivered as a composition. The composition may be formulated for parenteral, intramuscular, intracerebral, intravascular (including intravenous), subcutaneous, or transdermal administration. Compositions for parenteral administration may include sterile aqueous solutions which may also contain buffers, diluents and other suitable additives. The EVs may be formulated in a pharmaceutical composition, which may include pharmaceutically acceptable carriers, thickeners, diluents, buffers, preservatives, and other pharmaceutically acceptable carriers or excipients and the like in addition to the EVs.

Parenteral administration is generally characterized by injection, such as subcutaneously, intramuscularly, or intravenously. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include sodium chloride injection, ringers injection, isotonic dextrose injection, sterile water injection, dextrose and lactated ringers injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone.

Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment. The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations can be packaged in an ampoule, a vial or a syringe with a needle. All preparations for parenteral administration should be sterile, as is known and practiced in the art.

A therapeutically effective amount of composition is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs, and can generally be estimated based on EC50s found to be effective in vitro and in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection.

Preferably, the dose of a single intramuscular injection is in the range of about 5 to 20 μg. Preferably, the dose of single or multiple systemic injections is in the range of 10 to 100 mg/kg of body weight.

Due to construct clearance (and breakdown of any targeted molecule), the patient may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the patient undergo maintenance therapy, wherein the construct is administered in maintenance doses, ranging from 0.01 mg/kg to 100 mg per kg of body weight, once or more daily, to once every 20 years.

Also disclosed herein is a method to reduce exosomal release from the skin to reducing trafficking to the brain. For example, in some embodiments, neutral sphingomyelinase inhibitor GW4869 can be applied topically and/or via intradermal injection to reduce skin-exosome release.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: The Skin-Brain Axis in Alzheimer's Disease

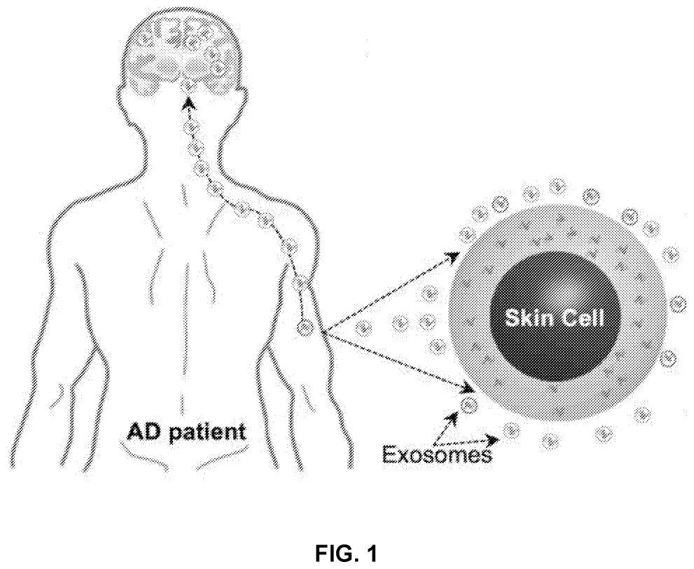

This Example seeks to elucidate the role of a potentially paradigm-shifting concept in Alzheimer's Disease (AD), the skin-brain axis, by evaluating the extent to which exosomes shed by skin cells can remotely modulate the onset and/or progression of AD, and whether exosome engineering approaches can lead to novel therapeutic strategies against this disease (FIG. 1). The proposed studies thus target a number of focus areas of high interest in AD research and care, including defining the contribution of non-neuronal tissues to neurodegeneration, and elucidating disease mechanisms that may point to novel avenues for intervention. While significant progress has been made towards understanding the biochemistry, genetics and pathology of AD, there are still major knowledge gaps in the temporal sequence of cellular and molecular events underlying onset or progression (Selkoe D. Ann Intern Med, 140(8):627-638). This Example studies, for the first time, whether AD can be triggered, worsened, or even treated, via remote cues emanating from the skin. As such, the work proposed herein is fundamentally innovative and potentially transformative.

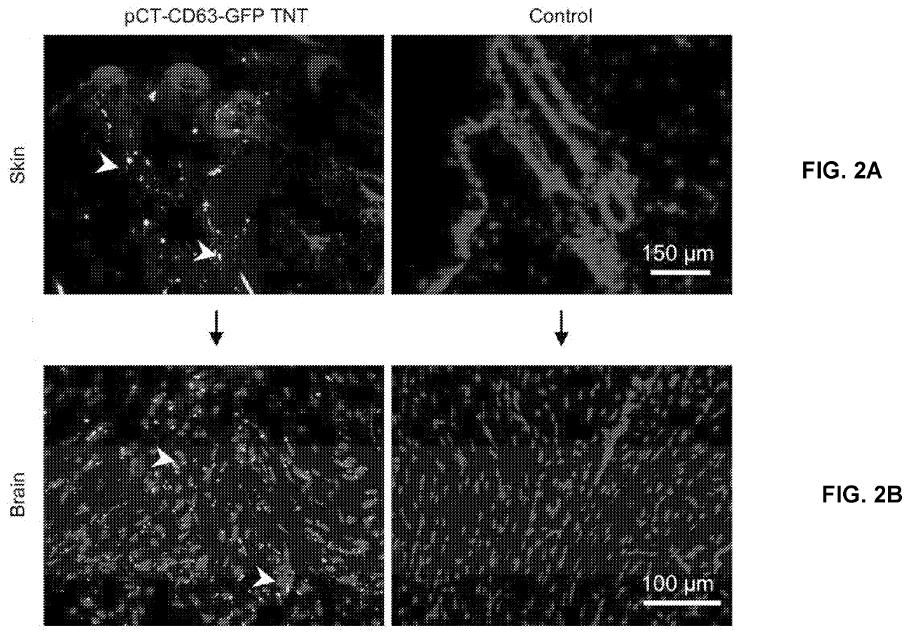

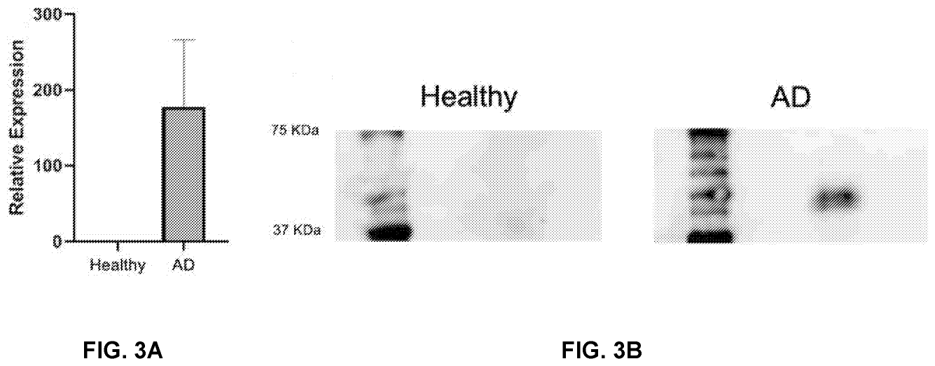

AD is the most common form of dementia currently affecting more than 5.8 million Americans, and expected to affect around 13.8 million by 2050 (Gaugler J, et al. ALZHEIMERS & DEMENTIA 2019, 15(3):321-387). AD is driven by the accumulation of plaques (i.e., aggregates of amyloid beta protein or Aβ) and tangles (i.e., aggregates of tau protein) within the brain (Small S A, et al. Neuron 2008, 60(4):534-542), which contribute to a profuse loss of synapses and neurons, partially mediated by inflammatory responses, eventually leading to a decline in mental abilities (Navarro Garrido V, et al. Frontiers in aging neuroscience 2018, 10:140; Mukhin V, et al. Neuroscience and Behavioral Physiology 2017, 47(5):508-516). Interestingly, the skin of AD patients has also been reported to harbor “deposits” of tau and amyloid (Bloom G S. JAMA neurology 2014, 71(4):505-508; Rodriguez-Leyva I, et al. J Mol Biomark Diagn S 2015, 6:005-010.4172; Jong Y-J I, et al. The FASEB journal 2003, 17(15):2319-2321; Okada A, et al. Dementia and Geriatric Cognitive Disorders 1994, 5(1):55-56). However, no clear functional role has been established for such deposits in the development or progression of AD. It was discovered in healthy mice that the skin can dispatch signals to the brain in the form of exosomes (FIG. 2), and that exosomes derived from the skin of mouse models of AD contain neurotoxic cargo that could potentially be impacting the progression of this disease (FIG. 3). Exosomes are cell-derived vesicles that play a crucial role in mediating cell-cell communications under both healthy and pathological conditions (S ELA, et al. Nat Rev Drug Discov 2013, 12(5):347-357; Ricklefs F, et al. Cancer Res 2016, 76(10):2876-2881; Hall J, et al. Cell Mol Neurobiol 2016, 36(3):417-427). It is hypothesize that: (1) exosomes derived from the skin could be cumulatively carrying neurotoxic cargo to the brain and contributing to the onset and/or progression of AD (i.e., skin-brain axis), and that (2) exosome engineering approaches can potentially be used as a novel therapeutic strategy against AD, using the skin as a “window” to the brain.

### Example 2: Characterization of Extracellular Vesicles from the Skin in the Progression of Alzheimer's Disease

Results

Differential Expression and Pathway Analyses of 3×Tg-AD Skin-Derived EVs Identified Changes in mRNA Expression and Pathways Related to Calcium Signaling

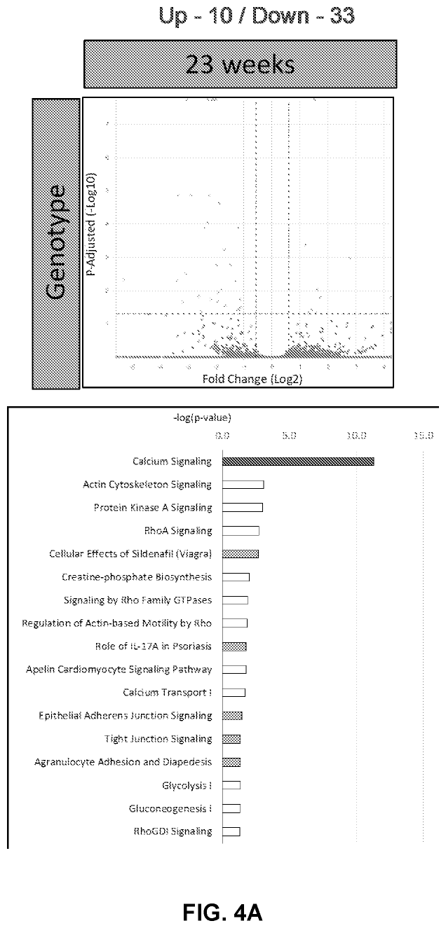

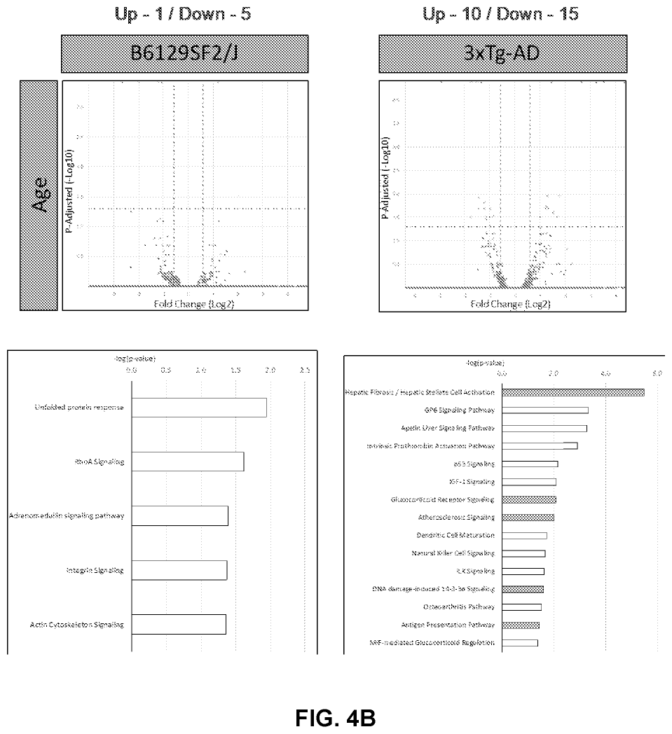

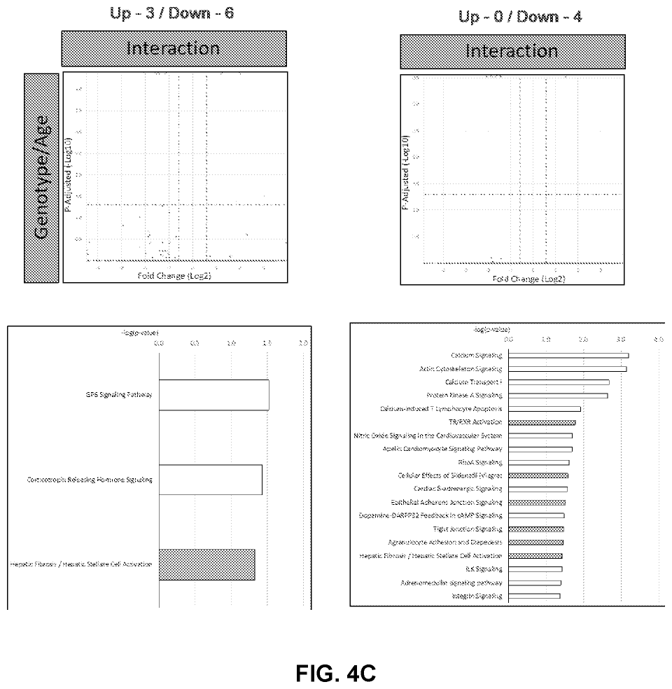

In order to characterize the neurodegenerative potential of skin-derived EVs within the context of AD, a broad characterization of differences in mRNA expression in 3×Tg-AD and B6129SF2/J skin-derived EVs was conducted at 10 and 23 weeks of age (n=3). This was accomplished through the use of RNA-seq and subsequent differential expression analysis. The differentially expressed genes from each comparison were subsequently filtered for a log fold change ±1.5 and adjusted p-value of 0.05 (FIG. 4). Ingenuity pathway analysis (IPA) was performed on resulting lists of differentially expressed genes in order to identify changes in canonical pathways and genes associated with specific diseases and/or functions (FIG. 4).

Differences in Extracellular Vesicles RNA-Content as a Function of Disease Genotype

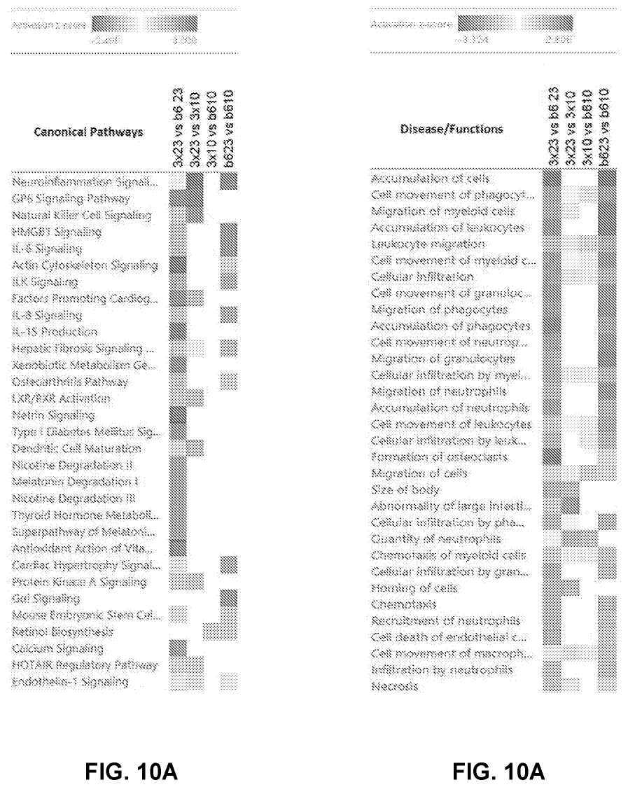

In order to determine the effect that the AD genotype has on changes in mRNA expression that occur within skin-derived EVs, the differential expression results from both 3×Tg-AD 10 week vs. B6129SF2/J 10 weeks and 3×Tg-AD 23 weeks vs. B6129SF2/J 23 weeks were taken into consideration. The 10 week comparison was considered in this case in order to evaluate the differences between 3×Tg-AD and control skin-derived EVs at a relatively early time point in disease progression compared to 23 weeks. This was done in an effort to establish a “baseline” to compare with changes that occur with disease progression found in the 3×Tg-AD 23 weeks vs. B6129SF2/J 23 weeks comparison. For the 3×Tg-AD compared with B6129SF2/J at 10 weeks 91 genes were upregulated and 79 were downregulated. The 3×Tg-AD 23 weeks vs. B6129SF2/J 23 weeks comparison was also considered here in order to evaluate how changes in mRNA expression occur at later stages in disease progression. The differential expression results revealed the upregulation of 240 genes and downregulation of 426 genes within this comparison (Table 1). In regards to pathways, calcium signaling showed the greatest amount of dysregulation within this comparison (−log p-value=11.261) (FIG. 10A, 10B). Furthermore, the dysregulation of calcium signaling pathways within the 3×Tg-AD 23 weeks vs. B6129SF2/J 23 weeks group represented the greatest magnitude of dysregulation relative to other dysregulated pathways found among all comparison groups in this study. A total of ten genes implicated with calcium signaling were found to be downregulated in the 23 week 3×Tg-AD mice. Of all the genes found to be differentially expressed within this dataset, the upregulation of S100 calcium-binding protein A9 (S100A9) (Log 2FoldChange=6.155) was found to be of the greatest magnitude. The change in expression of this gene in particular is interesting given the fact that this upregulation is related to amyloid plaque accumulation within the AD brain (Wang, C. et al. Acta Neuropathol. 2014 127:507-522). Other dysregulated pathways in this comparison included actin cytoskeleton signaling, protein kinase A signaling, and RhoA signaling.

TABLE 1

| Name | Description | log2FoldChange | pValue | Padj |
|---|---|---|---|---|
| Rn45s | Rn45s | −2.01655 | 1.62E−13 | 1.23E−09 |
| Mylpf | Mylpf | −3.05061 | 6.09E−12 | 2.30E−08 |
| Neb | Neb | −2.29724 | 5.31E−11 | 1.33E−07 |
| Rps3a1 | Rps3a1 | 1.501043 | 8.98E−11 | 1.69E−07 |
| Acta1 | Acta1 | −2.02599 | 5.13E−10 | 7.74E−07 |
| Atp2a1 | Atp2a1 | −2.12498 | 8.98E−10 | 1.13E−06 |
| Ckm | Ckm | −2.08923 | 1.25E−09 | 1.35E−06 |
| Ttn | Ttn | −2.15305 | 3.94E−09 | 3.72E−06 |
| Rnu11 | Rnu11 | −2.90544 | 1.79E−08 | 1.37E−05 |
| Myh2 | Myh2 | −3.35743 | 1.81E−08 | 1.37E−05 |
| Xirp2 | Xirp2 | −2.23579 | 2.04E−08 | 1.4E−05 |
| Lars2 | Lars2 | −1.90255 | 3.81E−08 | 2.39E−05 |
| Tnnc2 | Tnnc2 | −2.3053 | 1.55E−07 | 8.99E−05 |
| Dpt | Dpt | −1.21916 | 2.47E−07 | 0.000131 |
| Eno3 | Eno3 | −2.41246 | 2.61E−07 | 0.000131 |
| Elovl4 | Elovl4 | 1.185551 | 9.03E−07 | 0.000426 |
| Des | Des | −1.70727 | 1.31E−06 | 0.000583 |
| Nr1d1 | Nr1d1 | 2.761581 | 2.47E−06 | 0.001037 |
| Tpm2 | Tpm2 | −1.48427 | 4.36E−06 | 0.001675 |
| Tnnt3 | Tnnt3 | −1.97564 | 4.44E−06 | 0.001675 |
| Jph2 | Jph2 | −3.09823 | 8.63E−06 | 0.0031 |
| Smtnl1 | Smtnl1 | −5.31377 | 1.35E−05 | 0.00464 |
| Fbn1 | Fbn1 | −1.31336 | 1.59E−05 | 0.005109 |
| Fmod | Fmod | −3.39621 | 1.62E−05 | 0.005109 |
| Serpina3j | Serpina3j | 1.61635 | 3.19E−05 | 0.009404 |
| Tnni2 | Tnni2 | −1.82731 | 3.24E−05 | 0.009404 |
| Cmya5 | Cmya5 | −1.94162 | 4.03E−05 | 0.011254 |
| Hspb6 | Hspb6 | −2.6271 | 5.46E−05 | 0.014705 |
| Malat1 | Malat1 | 1.714919 | 6.25E−05 | 0.01627 |
| S100a9 | S100a9 | 6.155012 | 6.53E−05 | 0.016425 |
| Slc25a4 | Slc25a4 | −1.13994 | 7.84E−05 | 0.018533 |
| Trdn | Trdn | −2.19358 | 7.86E−05 | 0.018533 |
| Hfe2 | Hfe2 | −6.1621 | 9.04E−05 | 1 |
| Mybpc1 | Mybpc1 | −1.94276 | 9.48E−05 | 0.021676 |
| Casq1 | Casq1 | −3.2052 | 0.000101 | 0.022364 |
| Ryr1 | Ryr1 | −1.67613 | 0.000134 | 0.028896 |
| Car3 | Car3 | −1.12579 | 0.000155 | 0.032546 |
| Clec3b | Clec3b | −1.23964 | 0.000186 | 0.037767 |
| Gas6 | Gas6 | −1.18604 | 0.00019 | 0.037767 |
| Fbxo40 | Fbxo40 | −4.84696 | 0.000192 | 1 |
| Slc38a2 | Slc38a2 | 1.414986 | 0.000203 | 0.039271 |
| Myo18b | Myo18b | −2.53696 | 0.000222 | 0.04188 |
| BC100530 | BC100530 | 4.041777 | 0.000267 | 0.047601 |
| Lor | Lor | 1.54947 | 0.000268 | 0.047601 |
| Kdm6b | Kdm6b | 1.296562 | 0.000271 | 0.047601 |
| Myh6 | Myh6 | −4.42134 | 0.000293 | 0.050226 |
| Akr1e1 | Akr1e1 | −3.81294 | 0.000346 | 0.058098 |
| Cacna1s | Cacna1s | −2.48906 | 0.000355 | 0.058269 |
| Ogn | Ogn | −1.49121 | 0.000445 | 0.071395 |
| Spon2 | Spon2 | −2.5188 | 0.000468 | 0.073571 |
| Fhl1 | Fhl1 | −1.20607 | 0.000503 | 0.077438 |
| Icam1 | Icam1 | 2.107226 | 0.000563 | 0.084925 |
| Myoc | Myoc | −2.1158 | 0.000648 | 0.094972 |
| Rnase2b | Rnase2b | 5.186592 | 0.000654 | 0.094972 |
| Mb | Mb | −2.06322 | 0.000691 | 0.096864 |
| Mxd1 | Mxd1 | 2.67428 | 0.000693 | 0.096864 |
| Gbp2b | Gbp2b | −5.1882 | 0.000762 | 1 |
| Dhcr24 | Dhcr24 | 1.218208 | 0.000783 | 0.105503 |
| Mybph | Mybph | −3.34594 | 0.000811 | 1 |
| Igfbp6 | Igfbp6 | −1.19814 | 0.000837 | 0.110771 |
| Alpk3 | Alpk3 | −2.44193 | 0.000884 | 0.114999 |
| Gltp | Gltp | 1.225324 | 0.000906 | 0.11589 |
| Paqr7 | Paqr7 | 3.599479 | 0.000953 | 0.118575 |
| Krt6b | Krt6b | 2.660958 | 0.001009 | 0.12263 |
| Nr1d2 | Nr1d2 | 2.299411 | 0.001024 | 0.12263 |
| Dkk2 | Dkk2 | −1.78745 | 0.001045 | 0.123218 |
| Igfn1 | Igfn1 | −4.42739 | 0.001103 | 1 |
| Hydin | Hydin | −5.08037 | 0.001109 | 1 |
| Gm13177 | Gm13177 | 3.548479 | 0.001142 | 0.132556 |
| Mypn | Mypn | −2.48279 | 0.001171 | 0.13315 |
| Cdsn | Cdsn | 1.781493 | 0.001199 | 0.13315 |
| Myl1 | Myl1 | −1.60128 | 0.0012 | 0.13315 |
| Casc1 | Casc1 | −5.71507 | 0.001204 | 1 |
| Dbp | Dbp | 4.922612 | 0.001309 | 0.142415 |
| Clca2 | Clca2 | 1.800484 | 0.001321 | 0.142415 |
| Tcea3 | Tcea3 | −3.15101 | 0.00133 | 1 |
| Anxa3 | Anxa3 | −1.43393 | 0.001353 | 0.143766 |
| Hrc | Hrc | −3.07614 | 0.001398 | 1 |
| L2hgdh | L2hgdh | −2.64147 | 0.00156 | 0.163548 |
| Spp1 | Spp1 | 5.06403 | 0.001686 | 0.174237 |
| Arid5b | Arid5b | 1.782864 | 0.001795 | 0.178688 |
| Tpm1 | Tpm1 | −1.0815 | 0.0018 | 0.178688 |
| Pcf11 | Pcf11 | 1.713716 | 0.001829 | 0.179209 |
| Scarna2 | Scarna2 | 4.421738 | 0.001872 | 0.181073 |
| Col3a1 | Col3a1 | −1.04751 | 0.001967 | 0.187904 |
| Pttg1 | Pttg1 | −4.96692 | 0.002005 | 1 |
| Asb2 | Asb2 | −3.12394 | 0.002026 | 0.188355 |
| Krt6a | Krt6a | 1.029594 | 0.002047 | 0.188355 |
| Dsc3 | Dsc3 | 1.241542 | 0.002163 | 0.196676 |
| Trim54 | Trim54 | −2.82606 | 0.002201 | 0.197744 |
| Baiap3 | Baiap3 | −4.27016 | 0.00223 | 1 |
| 2310002L09Rik | 2310002L09Rik | −4.38627 | 0.002253 | 1 |
| Tgfb2 | Tgfb2 | −2.60914 | 0.00233 | 0.203905 |
| Il1b | Il1b | 2.262838 | 0.002367 | 0.203905 |
| Cilp | Cilp | −1.1313 | 0.002394 | 0.203905 |
| Pfkm | Pfkm | −1.44563 | 0.002405 | 0.203905 |
| Cdr1 | Cdr1 | −3.756 | 0.002405 | 1 |
| Gm13483 | Gm13483 | −5.54958 | 0.002669 | 1 |
| Scn10a | Scn10a | −5.54958 | 0.002669 | 1 |
| Slc17a7 | Slc17a7 | −5.54958 | 0.002669 | 1 |
| Myh4 | Myh4 | −2.46444 | 0.002741 | 0.229835 |
| Camk2a | Camk2a | −4.22147 | 0.002761 | 1 |
| Cxcl2 | Cxcl2 | 3.921186 | 0.002785 | 0.230908 |
| Pclo | Pclo | −3.39092 | 0.00285 | 0.233753 |
| Mt1 | Mt1 | 1.020204 | 0.002978 | 0.240546 |
| Tnfaip3 | Tnfaip3 | 1.309719 | 0.002996 | 0.240546 |
| Cntn2 | Cntn2 | −4.19439 | 0.003052 | 1 |
| Klhl31 | Klhl31 | −2.47059 | 0.003162 | 0.246059 |
| Mstn | Mstn | −3.33973 | 0.003163 | 1 |
| Dsp | Dsp | 1.112861 | 0.003184 | 0.246059 |
| Pcolce2 | Pcolce2 | −1.47185 | 0.003196 | 0.246059 |
| Fam207a | Fam207a | −1.58746 | 0.003255 | 0.248099 |
| Tnnt1 | Tnnt1 | −3.22037 | 0.003428 | 1 |
| Asic1 | Asic1 | −5.57818 | 0.003442 | 1 |
| Tagap | Tagap | −5.57818 | 0.003442 | 1 |
| Asb5 | Asb5 | −2.45579 | 0.003446 | 0.260022 |
| Fxyd2 | Fxyd2 | −3.39095 | 0.003481 | 1 |
| Srl | Srl | −1.54532 | 0.003633 | 0.269433 |
| Ldb3 | Ldb3 | −1.78926 | 0.003642 | 0.269433 |
| Ryr3 | Ryr3 | −4.22302 | 0.003653 | 1 |
| Nrap | Nrap | −1.52443 | 0.00375 | 0.273009 |
| Mybpc2 | Mybpc2 | −1.44084 | 0.003775 | 0.273009 |
| Gse1 | Gse1 | 2.762572 | 0.003799 | 0.273009 |
| Abra | Abra | −3.49618 | 0.00389 | 1 |
| Ildr1 | Ildr1 | −4.89509 | 0.003899 | 1 |
| Smyd1 | Smyd1 | −4.15977 | 0.003926 | 1 |
| Hmox1 | Hmox1 | 1.779002 | 0.003929 | 0.279708 |
| Obscn | Obscn | −1.31582 | 0.003987 | 0.280198 |
| Grin1 | Grin1 | −5.52872 | 0.00399 | 1 |
| Stk36 | Stk36 | −5.52872 | 0.00399 | 1 |
| S100a8 | S100a8 | 3.31818 | 0.00401 | 0.280198 |
| Zfp560 | Zfp560 | −3.17168 | 0.004098 | 1 |
| Fsd2 | Fsd2 | −4.78529 | 0.004158 | 1 |
| Polq | Polq | −3.36578 | 0.004316 | 1 |
| Thbs4 | Thbs4 | −1.49677 | 0.004335 | 0.298691 |
| Gsdma | Gsdma | 4.478911 | 0.004644 | 1 |
| Krt80 | Krt80 | 1.815378 | 0.004816 | 0.327409 |
| 1-Mar | 1-Mar | −3.61266 | 0.00485 | 1 |
| Fibin | Fibin | −2.03958 | 0.004921 | 0.331529 |
| Krt7 | Krt7 | −4.82646 | 0.004968 | 1 |
| Pik3cg | Pik3cg | −2.62412 | 0.005037 | 0.336382 |
| Bcan | Bcan | −4.73606 | 0.005324 | 1 |
| Flnc | Flnc | −1.25649 | 0.005437 | 0.356777 |
| Ndufv2 | Ndufv2 | −1.20956 | 0.005578 | 0.362495 |
| Tacstd2 | Tacstd2 | 1.169254 | 0.00562 | 0.362495 |
| Myoz1 | Myoz1 | −1.97927 | 0.005686 | 0.36364 |
| Zxdb | Zxdb | −3.2303 | 0.00569 | 1 |
| Il11ra1 | Il11ra1 | −1.19363 | 0.006103 | 0.386975 |
| Neurl1a | Neurl1a | −4.02742 | 0.006141 | 1 |
| Rbfox1 | Rbfox1 | −4.02742 | 0.006141 | 1 |
| Cdh1 | Cdh1 | 1.218104 | 0.00646 | 0.405499 |
| Fam83g | Fam83g | 2.406272 | 0.006502 | 0.405499 |
| Snora23 | Snora23 | 2.277804 | 0.006696 | 0.410801 |
| Plet1 | Plet1 | 1.603181 | 0.006822 | 0.41518 |
| Zfp799 | Zfp799 | −2.82483 | 0.006958 | 1 |
| Taf15 | Taf15 | −1.04278 | 0.006962 | 0.420268 |
| Cstf1 | Cstf1 | −2.05162 | 0.007177 | 0.426454 |
| 3425401B19Rik | 3425401B19Rik | −4.0632 | 0.007332 | 1 |
| Larp7 | Larp7 | −1.63099 | 0.00748 | 0.435798 |
| Slfn4 | Slfn4 | 3.67866 | 0.007554 | 0.435798 |
| Gm13375 | Gm13375 | −1.40837 | 0.007566 | 0.435798 |
| Ppp1r3a | Ppp1r3a | −2.38748 | 0.007904 | 0.448068 |
| Mmp15 | Mmp15 | −1.99343 | 0.007957 | 0.448068 |
| A1cf | A1cf | −5.32309 | 0.008058 | 1 |
| Chst3 | Chst3 | −5.32309 | 0.008058 | 1 |
| F630111L10Rik | F630111L10Rik | −5.32309 | 0.008058 | 1 |
| Gbp11 | Gbp11 | −5.32309 | 0.008058 | 1 |
| Gm6583 | Gm6583 | −5.32309 | 0.008058 | 1 |
| Lrguk | Lrguk | −5.32309 | 0.008058 | 1 |
| Mcidas | Mcidas | −5.32309 | 0.008058 | 1 |
| Rnf182 | Rnf182 | −5.32309 | 0.008058 | 1 |
| Syt2 | Syt2 | −5.32309 | 0.008058 | 1 |
| Tsks | Tsks | −5.32309 | 0.008058 | 1 |
| Ugt1a1 | Ugt1a1 | 1.41649 | 0.008076 | 0.4514 |
| Mgme1 | Mgme1 | −3.51674 | 0.008096 | 1 |
| Dnah8 | Dnah8 | −3.06769 | 0.008214 | 1 |
| Dock10 | Dock10 | −1.79807 | 0.008441 | 0.46834 |
| Lama2 | Lama2 | −1.0285 | 0.008592 | 0.471436 |
| Arntl | Arntl | −1.80777 | 0.008622 | 0.471436 |
| Mitf | Mitf | 2.031193 | 0.008762 | 0.474532 |
| Fuca2 | Fuca2 | −2.05736 | 0.008804 | 0.474532 |
| Kif9 | Kif9 | −4.5795 | 0.008967 | 1 |
| Myl2 | Myl2 | −4.5795 | 0.008967 | 1 |
| Adamts9 | Adamts9 | 1.629535 | 0.008978 | 0.474686 |
| Jak2 | Jak2 | 1.332688 | 0.009181 | 0.474686 |
| Fndc5 | Fndc5 | −4.5801 | 0.009197 | 1 |
| S100b | S100b | −4.5801 | 0.009197 | 1 |
| Ces1d | Ces1d | 1.523836 | 0.009199 | 0.474686 |
| Pygm | Pygm | −1.38063 | 0.009325 | 0.474686 |
| Dnah5 | Dnah5 | −5.34894 | 0.009443 | 1 |
| Ncan | Ncan | −5.34894 | 0.009443 | 1 |
| Ppp1r3f | Ppp1r3f | −5.34894 | 0.009443 | 1 |
| Hcar2 | Hcar2 | 3.700267 | 0.009481 | 0.474686 |
| Nov | Nov | −1.95145 | 0.009619 | 0.474686 |
| Ugcg | Ugcg | 1.437981 | 0.009731 | 0.474686 |
| Arc | Arc | 1.69328 | 0.009775 | 0.474686 |
| Tcf7l1 | Tcf7l1 | 2.545233 | 0.009783 | 0.474686 |
| Tmem182 | Tmem182 | −4.62744 | 0.009826 | 1 |
| Ddhd1 | Ddhd1 | −1.27582 | 0.009854 | 0.474686 |
| A130010J15Rik | A130010J15Rik | 4.250029 | 0.009929 | 1 |
| Hps3 | Hps3 | −2.14534 | 0.009929 | 0.474686 |
| Rlim | Rlim | 1.218141 | 0.009939 | 0.474686 |
| Hdac9 | Hdac9 | −3.44531 | 0.010138 | 1 |
| Nalcn | Nalcn | −3.86922 | 0.010347 | 1 |
| Fbxo17 | Fbxo17 | −2.77379 | 0.010422 | 1 |
| Helb | Helb | −2.15889 | 0.010437 | 0.491963 |
| Cd93 | Cd93 | 2.239158 | 0.010496 | 0.491963 |
| Cyp2b23 | Cyp2b23 | −2.76826 | 0.010627 | 1 |
| Gdpd3 | Gdpd3 | 3.533623 | 0.010832 | 0.501441 |
| Slfn10-ps | Slfn10-ps | −2.3461 | 0.010877 | 1 |
| Trim72 | Trim72 | −3.08128 | 0.010949 | 1 |
| Abat | Abat | −3.07088 | 0.011126 | 1 |
| Nup37 | Nup37 | −2.6722 | 0.01115 | 1 |
| Zc3h12c | Zc3h12c | 3.224777 | 0.011196 | 0.512034 |
| Kat2b | Kat2b | 1.513738 | 0.011482 | 0.520907 |
| Agpat6 | Agpat6 | −1.19519 | 0.011583 | 0.520907 |
| Vma21 | Vma21 | −1.49647 | 0.011597 | 0.520907 |
| Syn1 | Syn1 | −4.05181 | 0.011635 | 1 |
| Pitrm1 | Pitrm1 | −1.2312 | 0.011835 | 0.525516 |
| Igfbp2 | Igfbp2 | −1.75395 | 0.011839 | 0.525516 |
| Pvalb | Pvalb | −2.84838 | 0.012106 | 0.528142 |
| Nfkbiz | Nfkbiz | 1.146794 | 0.01219 | 0.528142 |
| Hist1h2ab | Hist1h2ab | −1.91852 | 0.012196 | 0.528142 |
| Actn3 | Actn3 | −2.19786 | 0.012226 | 0.528142 |
| Myo5b | Myo5b | 4.162 | 0.012229 | 1 |
| Dsc1 | Dsc1 | 1.447436 | 0.012282 | 0.528142 |
| Sowahc | Sowahc | 1.430243 | 0.012364 | 0.528142 |
| Sptbn2 | Sptbn2 | 1.542313 | 0.012458 | 0.528142 |
| Dna2 | Dna2 | −3.17686 | 0.012544 | 1 |
| Eomes | Eomes | −4.5388 | 0.012569 | 1 |
| Otog | Otog | −4.5388 | 0.012569 | 1 |
| Sike1 | Sike1 | −1.68526 | 0.012647 | 0.53018 |
| Ush1g | Ush1g | −4.53946 | 0.012657 | 1 |
| Muc19 | Muc19 | −3.84302 | 0.012823 | 1 |
| Mroh4 | Mroh4 | −4.54073 | 0.012828 | 1 |
| Rbm46 | Rbm46 | −4.54073 | 0.012828 | 1 |
| Rimbp3 | Rimbp3 | −4.54073 | 0.012828 | 1 |
| Gcnt2 | Gcnt2 | 2.429269 | 0.012986 | 0.541132 |
| Myh7 | Myh7 | −5.56409 | 0.012991 | 1 |
| Cxcl14 | Cxcl14 | −1.1179 | 0.013051 | 0.541132 |
| Tep1 | Tep1 | −1.39784 | 0.013279 | 0.544806 |
| Myom2 | Myom2 | −1.6264 | 0.013284 | 0.544806 |
| Stfa3 | Stfa3 | 4.052464 | 0.01331 | 1 |
| Chil3 | Chil3 | 4.107292 | 0.013367 | 1 |
| Sema7a | Sema7a | 3.473088 | 0.013394 | 0.546317 |
| Vsx2 | Vsx2 | −3.85519 | 0.013427 | 1 |
| Nfkb1 | Nfkb1 | 1.021191 | 0.0135 | 0.547695 |
| Ovol1 | Ovol1 | 1.695461 | 0.013727 | 0.553944 |
| Acss1 | Acss1 | −2.20503 | 0.013733 | 1 |
| Zfp719 | Zfp719 | −2.20503 | 0.013733 | 1 |
| Prr12 | Prr12 | −1.24287 | 0.013922 | 0.555808 |
| Myom3 | Myom3 | −2.21099 | 0.014012 | 1 |
| Arrdc4 | Arrdc4 | 1.428118 | 0.01403 | 0.555808 |
| H19 | H19 | −1.19307 | 0.014068 | 0.555808 |
| Dmpk | Dmpk | −1.54205 | 0.014308 | 0.558444 |
| Sik1 | Sik1 | 1.050553 | 0.014366 | 0.558444 |
| Elovl6 | Elovl6 | 1.1129 | 0.014431 | 0.558444 |
| Gspt2 | Gspt2 | −3.37355 | 0.014536 | 1 |
| Radil | Radil | −2.24283 | 0.014635 | 1 |
| Mfng | Mfng | −2.01299 | 0.014714 | 0.566507 |
| Atp6v1b2 | Atp6v1b2 | 1.490241 | 0.014899 | 0.570528 |
| Etv3 | Etv3 | 1.418816 | 0.01497 | 0.570528 |
| Rnasek | Rnasek | −1.46015 | 0.015284 | 0.572815 |
| Schip1 | Schip1 | −2.06398 | 0.015419 | 0.572815 |
| Epha2 | Epha2 | 1.49251 | 0.015546 | 0.572815 |
| 2310003H01Rik | 2310003H01Rik | 3.995093 | 0.015609 | 1 |
| Saa3 | Saa3 | 3.600453 | 0.015624 | 0.572815 |
| Mfap4 | Mfap4 | −1.14363 | 0.015827 | 0.57357 |
| Slc38a3 | Slc38a3 | −3.3972 | 0.016002 | 1 |
| Stfa1 | Stfa1 | 3.37521 | 0.01611 | 0.5769 |
| Mmp25 | Mmp25 | −2.78755 | 0.016181 | 1 |
| Zgrf1 | Zgrf1 | −2.78755 | 0.016181 | 1 |
| Epha4 | Epha4 | 2.349317 | 0.016198 | 0.5769 |
| Mdm2 | Mdm2 | 1.427786 | 0.016251 | 0.5769 |
| Lphn3 | Lphn3 | −2.10189 | 0.016412 | 0.57873 |
| Arid2 | Arid2 | 1.602695 | 0.01651 | 0.579463 |
| Map2k3 | Map2k3 | 1.182899 | 0.016708 | 0.583686 |
| Tgfbr1 | Tgfbr1 | −1.2562 | 0.016795 | 0.584035 |
| Ckap2 | Ckap2 | −2.06533 | 0.016899 | 1 |
| Rilpl1 | Rilpl1 | −1.61429 | 0.016999 | 0.588149 |
| Srgn | Srgn | 1.808823 | 0.017069 | 0.588149 |
| Duox1 | Duox1 | −2.44034 | 0.017082 | 1 |
| Cadps2 | Cadps2 | −2.08184 | 0.017216 | 1 |
| Wdr8 | Wdr8 | −2.43287 | 0.017249 | 1 |
| Vav2 | Vav2 | 3.955594 | 0.017574 | 1 |
| Gna15 | Gna15 | −1.87521 | 0.017631 | 0.595922 |
| Tef | Tef | 1.436745 | 0.017734 | 0.595922 |
| Ifit3 | Ifit3 | −1.48969 | 0.017767 | 0.595922 |
| Limch1 | Limch1 | −1.43699 | 0.017769 | 0.595922 |
| Mmp17 | Mmp17 | −2.97939 | 0.017891 | 1 |
| Rasgrp3 | Rasgrp3 | −2.80942 | 0.018044 | 1 |
| B3glct | B3glct | −1.90724 | 0.018131 | 0.602701 |
| Pnpla1 | Pnpla1 | 4.079731 | 0.018201 | 1 |
| Sfi1 | Sfi1 | −1.75378 | 0.018213 | 0.602786 |
| Mgl2 | Mgl2 | −1.17383 | 0.018533 | 0.610686 |
| Rad9a | Rad9a | 4.060476 | 0.018548 | 1 |
| Rap1gap | Rap1gap | −3.34707 | 0.018561 | 1 |
| Utp23 | Utp23 | −2.28598 | 0.018698 | 1 |
| Tbl1x | Tbl1x | 1.431094 | 0.019608 | 0.632581 |
| Wdr60 | Wdr60 | −1.93055 | 0.019762 | 0.632581 |
| Casp4 | Casp4 | 3.328155 | 0.019764 | 0.632581 |
| Rab11fip1 | Rab11fip1 | 1.848593 | 0.019865 | 0.632581 |
| Rbm20 | Rbm20 | −2.27054 | 0.019878 | 1 |
| Ptpn9 | Ptpn9 | 2.004286 | 0.019936 | 0.632581 |
| Snora64 | Snora64 | 2.448604 | 0.020094 | 0.634447 |
| Nrip1 | Nrip1 | 1.873151 | 0.020226 | 0.635942 |
| Glp1r | Glp1r | −3.6841 | 0.020368 | 1 |
| 9530051G07Rik | 9530051G07Rik | −4.34401 | 0.020416 | 1 |
| Neurod1 | Neurod1 | −4.34401 | 0.020416 | 1 |
| Sh3gl3 | Sh3gl3 | −4.34401 | 0.020416 | 1 |
| Tex16 | Tex16 | −4.34401 | 0.020416 | 1 |
| B0C68157 | B0C68157 | −4.34445 | 0.020601 | 1 |
| Dennd6b | Dennd6b | −4.34445 | 0.020601 | 1 |
| Setmar | Setmar | −4.34445 | 0.020601 | 1 |
| Gtf2ird2 | Gtf2ird2 | −2.80274 | 0.020638 | 1 |
| Shank3 | Shank3 | 1.972941 | 0.020738 | 0.64932 |
| Gm7120 | Gm7120 | −4.34526 | 0.020945 | 1 |
| Atp8b3 | Atp8b3 | −4.3961 | 0.021016 | 1 |
| Shisa4 | Shisa4 | −4.3961 | 0.021016 | 1 |
| Sqle | Sqle | 1.394828 | 0.02114 | 0.650452 |
| Klhl41 | Klhl41 | −1.59091 | 0.021162 | 0.650452 |
| Tmem198 | Tmem198 | −3.27054 | 0.021219 | 1 |
| 1810013L24Rik | 1810013L24Rik | 1.206171 | 0.021301 | 0.650452 |
| Slc1a3 | Slc1a3 | 2.008642 | 0.021385 | 0.650452 |
| Tsc22d2 | Tsc22d2 | 1.331276 | 0.021439 | 0.650452 |
| Emp2 | Emp2 | 1.028533 | 0.021489 | 0.650452 |
| F3 | F3 | 1.873621 | 0.02155 | 0.650452 |
| Hmga2-ps1 | Hmga2-ps1 | −2.35729 | 0.021774 | 1 |
| Xirp1 | Xirp1 | −1.5126 | 0.021848 | 0.656495 |
| Rabl2 | Rabl2 | −2.69903 | 0.022077 | 1 |
| Speg | Speg | −1.41565 | 0.022142 | 0.656495 |
| Mon1a | Mon1a | 3.783599 | 0.022223 | 1 |
| Dtna | Dtna | −2.07759 | 0.022342 | 1 |
| Agap3 | Agap3 | 1.72541 | 0.022366 | 0.656495 |
| Phactr4 | Phactr4 | 1.794199 | 0.022398 | 0.656495 |
| Piezo1 | Piezo1 | 1.347808 | 0.022456 | 0.656495 |
| Spta1 | Spta1 | −2.36328 | 0.022494 | 1 |
| Foxp2 | Foxp2 | −2.3492 | 0.022496 | 1 |
| Atp2b4 | Atp2b4 | 1.165353 | 0.022514 | 0.656495 |
| Crtc2 | Crtc2 | 1.766244 | 0.022651 | 0.656495 |
| Gpr34 | Gpr34 | −3.22816 | 0.022711 | 1 |
| Lama5 | Lama5 | 1.399448 | 0.022714 | 0.656495 |
| Cdh5 | Cdh5 | 1.671799 | 0.022787 | 0.656495 |
| Hrnr | Hrnr | 3.064058 | 0.022842 | 0.656495 |
| Islr2 | Islr2 | −2.96566 | 0.022845 | 1 |
| Plk2 | Plk2 | 1.217302 | 0.022954 | 0.656495 |
| Celsr1 | Celsr1 | 1.48351 | 0.023099 | 0.656495 |
| Snord15a | Snord15a | 2.736879 | 0.023222 | 0.656495 |
| Nhlrc1 | Nhlrc1 | −2.51226 | 0.023463 | 1 |
| St3gal3 | St3gal3 | −1.93522 | 0.023605 | 0.66465 |
| Mapk13 | Mapk13 | 3.237848 | 0.023762 | 0.666563 |
| Scarna13 | Scarna13 | 3.345444 | 0.024075 | 0.670364 |
| Ccl4 | Ccl4 | 3.764646 | 0.024182 | 1 |
| Spint2 | Spint2 | 1.996497 | 0.024366 | 0.673514 |
| Jpx | Jpx | −3.73683 | 0.024391 | 1 |
| Ankrd26 | Ankrd26 | −2.49433 | 0.024427 | 1 |
| Efna1 | Efna1 | 3.767076 | 0.024515 | 1 |
| Ggct | Ggct | 2.084914 | 0.024539 | 0.674774 |
| H2-T23 | H2-T23 | 1.345586 | 0.024591 | 0.674774 |
| Myot | Myot | −3.18173 | 0.024744 | 0.676526 |
| Pcca | Pcca | −1.67413 | 0.02485 | 0.676954 |
| Cacna2d2 | Cacna2d2 | −2.71639 | 0.025064 | 1 |
| Kpna7 | Kpna7 | −3.84452 | 0.025112 | 1 |
| Msh5 | Msh5 | −3.84452 | 0.025112 | 1 |
| Batf | Batf | −2.91086 | 0.025266 | 1 |
| Lgals4 | Lgals4 | 3.909103 | 0.025318 | 1 |
| Fbxo30 | Fbxo30 | 1.684031 | 0.025329 | 0.677196 |
| Synpo2 | Synpo2 | −1.24475 | 0.025335 | 0.677196 |
| 4930563E22Rik | 4930563E22Rik | −5.06583 | 0.025432 | 1 |
| C730027H18Rik | C730027H18Rik | −5.06583 | 0.025432 | 1 |
| Fbxo27 | Fbxo27 | −5.06583 | 0.025432 | 1 |
| Foxa2 | Foxa2 | −5.06583 | 0.025432 | 1 |
| Lrriq1 | Lrriq1 | −5.06583 | 0.025432 | 1 |
| Npy6r | Npy6r | −5.06583 | 0.025432 | 1 |
| Pld5 | Pld5 | −5.06583 | 0.025432 | 1 |
| Pth2r | Pth2r | −5.06583 | 0.025432 | 1 |
| Rab3c | Rab3c | −5.06583 | 0.025432 | 1 |
| Scgb3a2 | Scgb3a2 | −5.06583 | 0.025432 | 1 |
| Slc17a3 | Slc17a3 | −5.06583 | 0.025432 | 1 |
| Slc6a7 | Slc6a7 | −5.06583 | 0.025432 | 1 |
| Spata31d1d | Spata31d1d | −5.06583 | 0.025432 | 1 |
| Srrm4os | Srrm4os | −5.06583 | 0.025432 | 1 |
| Tigd4 | Tigd4 | −5.06583 | 0.025432 | 1 |
| Tmco5 | Tmco5 | −5.06583 | 0.025432 | 1 |
| Vwa7 | Vwa7 | −5.06583 | 0.025432 | 1 |
| Gpr56 | Gpr56 | 1.238249 | 0.025463 | 0.677196 |
| Adssl1 | Adssl1 | −1.70656 | 0.0256 | 0.677196 |
| Pard6b | Pard6b | 3.268984 | 0.025672 | 0.677196 |
| Usp53 | Usp53 | 3.14222 | 0.025725 | 0.677196 |
| Itch | Itch | 1.421224 | 0.025873 | 0.677196 |
| Col24a1 | Col24a1 | −2.50778 | 0.025904 | 1 |
| Vipr1 | Vipr1 | −2.70191 | 0.025919 | 1 |
| Gm13178 | Gm13178 | −1.23643 | 0.025936 | 0.677196 |
| Lrrc39 | Lrrc39 | −2.99663 | 0.026033 | 1 |
| Cntnap1 | Cntnap1 | −3.6746 | 0.026048 | 1 |
| Myh3 | Myh3 | −3.6746 | 0.026048 | 1 |
| Slfn9 | Slfn9 | −2.22853 | 0.026208 | 1 |
| Slc16a6 | Slc16a6 | −1.99641 | 0.026336 | 1 |
| 4930431P03Rik | 4930431P03Rik | −5.08035 | 0.026407 | 1 |
| 9130204L05Rik | 9130204L05Rik | −5.08035 | 0.026407 | 1 |
| Ghsr | Ghsr | −5.08035 | 0.026407 | 1 |
| Icos | Icos | −5.08035 | 0.026407 | 1 |
| Slc26a3 | Slc26a3 | −5.08035 | 0.026407 | 1 |
| Trappc3l | Trappc3l | −5.08035 | 0.026407 | 1 |
| Unc5d | Unc5d | −5.08035 | 0.026407 | 1 |
| Pim1 | Pim1 | 1.132869 | 0.026447 | 0.685807 |
| Agtr1a | Agtr1a | −2.00912 | 0.026539 | 0.685835 |
| Tie1 | Tie1 | 3.145369 | 0.027 | 0.693399 |
| Dancr | Dancr | 2.543538 | 0.027169 | 0.693399 |
| Phkb | Phkb | −1.32101 | 0.027265 | 0.693399 |
| Ubl3 | Ubl3 | 1.277234 | 0.027447 | 0.693399 |
| Irf1 | Irf1 | 1.239416 | 0.027621 | 0.693399 |
| Smtn | Smtn | 1.552558 | 0.027743 | 0.693399 |
| Plk3 | Plk3 | 1.896446 | 0.027761 | 0.693399 |
| Efnb2 | Efnb2 | 1.333616 | 0.027784 | 0.693399 |
| Abca8b | Abca8b | −1.52081 | 0.027843 | 0.693399 |
| Lgals6 | Lgals6 | 3.863529 | 0.027994 | 1 |
| Tmppe | Tmppe | 3.690236 | 0.028044 | 1 |
| Pde4d | Pde4d | −1.43076 | 0.028053 | 0.694051 |
| Clec2i | Clec2i | −3.627 | 0.028247 | 1 |
| Gba | Gba | 1.900757 | 0.028366 | 0.69741 |
| Brwd1 | Brwd1 | 1.15313 | 0.028373 | 0.69741 |
| Smpx | Smpx | −2.23702 | 0.028509 | 1 |
| Sprr2h | Sprr2h | 3.844695 | 0.028562 | 1 |
| Fermt1 | Fermt1 | −1.60246 | 0.028682 | 0.698988 |
| Chd3os | Chd3os | 3.693416 | 0.02879 | 1 |
| 5-Mar | 5-Mar | −1.09371 | 0.028865 | 0.698988 |
| Pkia | Pkia | −1.34705 | 0.028881 | 0.698988 |
| Spock2 | Spock2 | −1.67084 | 0.028901 | 0.698988 |
| Rhebl1 | Rhebl1 | −3.087 | 0.028944 | 1 |
| Col22a1 | Col22a1 | −3.68719 | 0.029023 | 1 |
| Amot | Amot | −1.57915 | 0.029055 | 0.70047 |
| Pde4b | Pde4b | 1.469652 | 0.029184 | 0.701139 |
| 9030624J02Rik | 9030624J02Rik | 2.149677 | 0.029268 | 0.701139 |
| Pecam1 | Pecam1 | 2.274054 | 0.02942 | 0.702354 |
| Gsto1 | Gsto1 | 2.580653 | 0.029505 | 0.702354 |
| Gadl1 | Gadl1 | 3.917891 | 0.029719 | 1 |
| Lep | Lep | −1.18627 | 0.029802 | 0.704267 |
| Gabra3 | Gabra3 | −1.83279 | 0.030075 | 1 |
| Kdm2b | Kdm2b | −1.14038 | 0.030091 | 0.704267 |
| Fndc9 | Fndc9 | 3.778463 | 0.030118 | 1 |
| Alpk2 | Alpk2 | −2.64799 | 0.030244 | 1 |
| Ptgs2 | Ptgs2 | 1.123744 | 0.030378 | 0.704267 |
| Tob2 | Tob2 | 1.061583 | 0.030405 | 0.704267 |
| Ccdc61 | Ccdc61 | −2.16224 | 0.030464 | 1 |
| Fgf10 | Fgf10 | −1.3767 | 0.030589 | 0.704267 |
| Scn4a | Scn4a | −1.77772 | 0.030621 | 0.704267 |
| AU018091 | AU018091 | −3.24906 | 0.03087 | 1 |
| Sumf1 | Sumf1 | −1.32746 | 0.031002 | 0.704267 |
| Mir6516 | Mir6516 | 2.69143 | 0.031046 | 0.704267 |
| Abca12 | Abca12 | 2.429326 | 0.0311 | 0.704267 |
| Sdr16c5 | Sdr16c5 | −1.70895 | 0.031203 | 0.704267 |
| Txlnb | Txlnb | −1.70895 | 0.031203 | 0.704267 |
| Snord22 | Snord22 | 2.031569 | 0.031205 | 0.704267 |
| Palm3 | Palm3 | −2.69503 | 0.031205 | 1 |
| Itsn2 | Itsn2 | 1.313373 | 0.031232 | 0.704267 |
| Pmm1 | Pmm1 | −1.72852 | 0.031246 | 0.704267 |
| Coq10b | Coq10b | 2.524455 | 0.031396 | 0.704267 |
| Aga | Aga | −2.15299 | 0.031421 | 1 |
| Selp | Selp | 2.510309 | 0.031429 | 0.704267 |
| Mfap5 | Mfap5 | −1.21148 | 0.031444 | 0.704267 |
| Slc16a13 | Slc16a13 | 3.624454 | 0.031754 | 1 |
| Gtf2h3 | Gtf2h3 | 3.630426 | 0.031797 | 1 |
| Zswim4 | Zswim4 | 1.29712 | 0.031885 | 0.70765 |
| Greb1 | Greb1 | −2.29316 | 0.032032 | 1 |
| Gna12 | Gna12 | 2.095262 | 0.032063 | 0.709514 |
| Ddit4l | Ddit4l | −2.15929 | 0.032358 | 1 |
| Cuedc2 | Cuedc2 | −1.40664 | 0.032499 | 0.712891 |
| Mrpl44 | Mrpl44 | −1.9592 | 0.032725 | 1 |
| Gm15800 | Gm15800 | 1.324911 | 0.032725 | 0.715783 |
| Ldlrad1 | LdIrad1 | −3.70237 | 0.03275 | 1 |
| Misp | Misp | −3.70237 | 0.03275 | 1 |
| Nphp4 | Nphp4 | −3.12695 | 0.032815 | 1 |
| Cd14 | Cd14 | 1.611754 | 0.032964 | 0.717388 |
| Pfkfb1 | Pfkfb1 | −3.18335 | 0.032993 | 1 |
| Slc41a3 | Slc41a3 | −2.67082 | 0.033061 | 1 |
| Herc4 | Herc4 | 1.230762 | 0.033215 | 0.718441 |
| Cadm4 | Cadm4 | −1.76013 | 0.033228 | 0.718441 |
| Scarf2 | Scarf2 | −1.84297 | 0.033354 | 1 |
| Ckmt2 | Ckmt2 | −2.99366 | 0.033391 | 1 |
| C030039L03Rik | C030039L03Rik | −2.84183 | 0.033489 | 1 |
| Fkbp5 | Fkbp5 | 1.15811 | 0.033735 | 0.726063 |
| Wnk2 | Wnk2 | 2.423433 | 0.033773 | 0.726063 |
| Hmces | Hmces | −2.06355 | 0.0339 | 1 |
| Zbtb9 | Zbtb9 | −2.28349 | 0.034116 | 1 |
| Hoxc10 | Hoxc10 | 3.630434 | 0.034295 | 1 |
| Ccnl1 | Ccnl1 | 1.204192 | 0.034301 | 0.732532 |
| Cldn4 | Cldn4 | 2.256268 | 0.034497 | 0.732532 |
| Ccl3 | Ccl3 | 3.615042 | 0.034552 | 1 |
| Eef2k | Eef2k | 1.190443 | 0.034698 | 0.733415 |
| Irg1 | Irg1 | 3.039383 | 0.034796 | 1 |
| Cpne7 | Cpne7 | −2.62108 | 0.034818 | 1 |
| Aqp1 | Aqp1 | 1.798313 | 0.034833 | 0.734209 |
| A930013F10Rik | A930013F10Rik | −2.86912 | 0.034971 | 1 |
| Exo1 | Exo1 | −2.86912 | 0.034971 | 1 |
| Csf3 | Csf3 | 3.106168 | 0.03499 | 0.734442 |
| Dsc2 | Dsc2 | 1.607391 | 0.03507 | 0.734442 |
| Trpm5 | Trpm5 | −3.63423 | 0.035175 | 1 |
| Fbxw17 | Fbxw17 | −2.09966 | 0.035232 | 1 |
| Tmem150b | Tmem150b | −3.21964 | 0.035438 | 1 |
| Ggps1 | Ggps1 | −1.6552 | 0.035505 | 0.738077 |
| Bhlhb9 | Bhlhb9 | −1.76813 | 0.03559 | 1 |
| Hs1bp3 | Hs1bp3 | 3.636764 | 0.035676 | 1 |
| Fam102a | Fam102a | 1.246918 | 0.035839 | 0.742969 |
| Pcyox1l | Pcyox1l | −3.05695 | 0.036333 | 1 |
| Fnip2 | Fnip2 | 1.469361 | 0.036498 | 0.754566 |
| Zfp637 | Zfp637 | 3.599414 | 0.036623 | 1 |
| Cpt1b | Cpt1b | −2.54241 | 0.036726 | 1 |
| Il31ra | Il31ra | −2.06238 | 0.036833 | 1 |
| C920009B18Rik | C920009B18Rik | −2.78645 | 0.037169 | 1 |
| Klk7 | Klk7 | 1.22907 | 0.037199 | 0.759678 |
| Sp140 | Sp140 | −1.4007 | 0.037249 | 0.759678 |
| Rnf122 | Rnf122 | 3.587858 | 0.03736 | 1 |
| Adamts17 | Adamts17 | −2.83093 | 0.037523 | 1 |
| Irf6 | Irf6 | 1.064384 | 0.038411 | 0.770105 |
| Podxl | Podxl | 1.931629 | 0.038508 | 0.770105 |
| Klb | Klb | 3.542937 | 0.038629 | 1 |
| Dusp1 | Dusp1 | 1.305675 | 0.038639 | 0.770105 |
| Dus1l | Dus1l | −1.46951 | 0.038656 | 0.770105 |
| Slc2a4 | Slc2a4 | −1.74691 | 0.038679 | 0.770105 |
| Pgr | Pgr | −3.53011 | 0.038919 | 0.771065 |
| Snora26 | Snora26 | 2.378358 | 0.039175 | 0.771065 |
| D430019H16Rik | D430019H16Rik | −3.53117 | 0.039189 | 1 |
| Fbxl15 | Fbxl15 | −2.42598 | 0.039203 | 1 |
| Adamtsl4 | Adamtsl4 | −1.46202 | 0.039315 | 0.771065 |
| Sbf1 | Sbf1 | 1.072082 | 0.03934 | 0.771065 |
| Ccdc71l | Ccdc71l | 1.265438 | 0.039542 | 0.771163 |
| Slc39a3 | Slc39a3 | −1.75205 | 0.039578 | 0.771163 |
| Col4a4 | Col4a4 | −1.52264 | 0.039652 | 0.771163 |
| Rasl10b | Rasl10b | −3.51489 | 0.039769 | 1 |
| Sspo | Sspo | −1.81154 | 0.039774 | 1 |
| Tmf1 | Tmf1 | 1.057854 | 0.039792 | 0.771902 |
| C030006K11Rik | C030006K11Rik | 3.684524 | 0.039868 | 1 |
| Fundc2 | Fundc2 | −1.0793 | 0.039927 | 0.772534 |
| Hccs | Hccs | −1.4279 | 0.040294 | 0.777634 |
| Olfml2a | Olfml2a | −3.48079 | 0.041139 | 1 |
| Nppb | Nppb | −3.27851 | 0.041216 | 1 |
| Plaur | Plaur | 2.35689 | 0.041319 | 0.783703 |
| Pld3 | Pld3 | 1.395109 | 0.041376 | 0.783703 |
| Hhipl1 | Hhipl1 | −3.01056 | 0.041516 | 1 |
| Dusp13 | Dusp13 | −4.39025 | 0.041519 | 1 |
| Gpr171 | Gpr171 | −4.39025 | 0.041519 | 1 |
| Igf2bp3 | Igf2bp3 | −4.39025 | 0.041519 | 1 |
| Sfxn4 | Sfxn4 | −4.39025 | 0.041519 | 1 |
| Adss | Adss | 1.318301 | 0.041548 | 0.783703 |
| Tollip | Tollip | 1.01268 | 0.041619 | 0.783703 |
| Lmtk2 | Lmtk2 | 1.442088 | 0.041759 | 0.783703 |
| Sele | Sele | 3.165771 | 0.041807 | 0.783703 |
| Nup62 | Nup62 | 1.365993 | 0.041908 | 0.783703 |
| Dusp5 | Dusp5 | 2.0537 | 0.042025 | 0.783703 |
| Csrp3 | Csrp3 | −1.06994 | 0.042043 | 0.783703 |
| Akap5 | Akap5 | −3.44167 | 0.042157 | 1 |
| Myo1a | Myo1a | −3.44167 | 0.042157 | 1 |
| Onecut2 | Onecut2 | −3.44167 | 0.042157 | 1 |
| Clec11a | Clec11a | −2.34205 | 0.042259 | 1 |
| Ssc5d | Ssc5d | −1.89547 | 0.042291 | 0.783703 |
| Faah | Faah | 2.957955 | 0.042309 | 0.783703 |
| Per2 | Per2 | 3.526256 | 0.04236 | 1 |
| Rfxap | Rfxap | 3.507042 | 0.042412 | 1 |
| Zfp131 | Zfp131 | 1.913723 | 0.042418 | 0.783703 |
| Ippk | Ippk | 2.15445 | 0.042477 | 0.783703 |
| Endov | Endov | −1.84361 | 0.042571 | 1 |
| Top3b | Top3b | −1.45725 | 0.042723 | 0.783703 |
| Szt2 | Szt2 | −1.39192 | 0.042965 | 0.783703 |
| Zbtb16 | Zbtb16 | 1.040903 | 0.042986 | 0.783703 |
| Aacs | Aacs | 1.280801 | 0.04303 | 0.783703 |
| BC024139 | BC024139 | −3.16878 | 0.043177 | 1 |
| Stx11 | Stx11 | 2.34188 | 0.043348 | 0.783703 |
| Stambpl1 | Stambpl1 | −1.95341 | 0.043398 | 0.783703 |
| Pcdh1 | Pcdh1 | 1.596894 | 0.043516 | 0.783703 |
| Trim45 | Trim45 | −3.40933 | 0.043535 | 1 |
| Ttll8 | Ttll8 | −3.40933 | 0.043535 | 1 |
| Il2rg | Il2rg | 3.500525 | 0.043641 | 1 |
| Serpina3h | Serpina3h | 3.51072 | 0.043661 | 1 |
| Sptbn4 | Sptbn4 | −2.68427 | 0.043699 | 1 |
| Alox8 | Alox8 | −3.23664 | 0.043821 | 1 |
| Lmod3 | Lmod3 | −3.23664 | 0.043821 | 1 |
| Arf2 | Arf2 | 1.577249 | 0.043936 | 0.787504 |
| Fosl1 | Fosl1 | 1.026545 | 0.044154 | 0.787665 |
| Yae1d1 | Yae1d1 | 3.490386 | 0.044193 | 1 |
| H2-Q4 | H2-Q4 | 1.396741 | 0.044804 | 0.791097 |
| Slc16a12 | Slc16a12 | −2.20982 | 0.044862 | 1 |
| Stoml1 | Stoml1 | −2.20982 | 0.044862 | 1 |
| Epdr1 | Epdr1 | −1.66756 | 0.044972 | 0.791097 |
| Bcl9l | Bcl9l | 1.099934 | 0.044997 | 0.791097 |
| Bpifc | Bpifc | 1.943582 | 0.045046 | 0.791097 |
| Nuak1 | Nuak1 | 1.746204 | 0.045062 | 0.791097 |
| Primpol | Primpol | −2.66591 | 0.045088 | 1 |
| Abcb1b | Abcb1b | −1.30393 | 0.045114 | 0.791097 |
| Abca17 | Abca17 | −4.12328 | 0.045115 | 1 |
| Abcc6 | Abcc6 | −4.12328 | 0.045115 | 1 |
| Cfi | Cfi | −4.12328 | 0.045115 | 1 |
| Slc30a3 | Slc30a3 | −4.12328 | 0.045115 | 1 |
| Tmem151b | Tmem151b | −4.12328 | 0.045115 | 1 |
| Mylk2 | Mylk2 | −1.55802 | 0.045204 | 0.791097 |
| Pde12 | Pde12 | 1.64205 | 0.045289 | 0.791097 |
| Lsr | Lsr | 1.711094 | 0.045425 | 0.791632 |
| C530008M17Rik | C530008M17Rik | −4.12315 | 0.045645 | 1 |
| Nap1l5 | Nap1l5 | −4.12315 | 0.045645 | 1 |
| Nos2 | Nos2 | −4.12315 | 0.045645 | 1 |
| P4ha3 | P4ha3 | −4.12315 | 0.045645 | 1 |
| E330033B04Rik | E330033B04Rik | −3.20835 | 0.045727 | 1 |
| Myo5c | Myo5c | −2.65689 | 0.045778 | 1 |
| 4931406P16Rik | 4931406P16Rik | 1.567154 | 0.045841 | 0.793379 |
| Ugt1a7c | Ugt1a7c | 1.119735 | 0.046131 | 0.79471 |
| Dedd2 | Dedd2 | 2.284704 | 0.046233 | 0.79471 |
| Cacna1b | Cacna1b | −3.05751 | 0.04639 | 1 |
| 2810417H13Rik | 2810417H13Rik | 3.518507 | 0.046436 | 1 |
| Acyr2b | Acyr2b | −2.60996 | 0.046537 | 1 |
| Hbegf | Hbegf | 1.522238 | 0.046556 | 0.798442 |
| Fbxw9 | Fbxw9 | 3.50676 | 0.046881 | 1 |
| Mfap3 | Mfap3 | 3.517178 | 0.046954 | 1 |
| Al661453 | Al661453 | 1.576707 | 0.047182 | 0.80143 |
| Fdx1 | Fdx1 | 3.469752 | 0.047194 | 1 |
| Ankrd55 | Ankrd55 | −3.04468 | 0.047228 | 1 |
| Imp3 | Imp3 | 1.995868 | 0.047231 | 0.80143 |
| Grhl3 | Grhl3 | 2.667311 | 0.047235 | 0.80143 |
| Map3k11 | Map3k11 | 1.298096 | 0.047347 | 0.80143 |
| Hdc | Hdc | 1.938371 | 0.047368 | 0.80143 |
| Dennd3 | Dennd3 | 3.535881 | 0.047741 | 1 |
| Gpd1l | Gpd1l | −1.33149 | 0.047752 | 0.804153 |
| Akap1 | Akap1 | −1.56552 | 0.047867 | 0.804153 |
| R3hcc1 | R3hcc1 | −1.23952 | 0.048011 | 0.804153 |
| Procr | Procr | 1.87423 | 0.048062 | 0.804153 |
| Adhfe1 | Adhfe1 | −2.29151 | 0.048167 | 1 |
| 3-Sep | 3-Sep | −1.86308 | 0.048452 | 1 |
| AY512915 | AY512915 | −4.07252 | 0.048462 | 1 |
| Catsperg2 | Catsperg2 | −4.07252 | 0.048462 | 1 |
| Cngb3 | Cngb3 | −4.07252 | 0.048462 | 1 |
| Dnaaf1 | Dnaaf1 | −4.07252 | 0.048462 | 1 |
| Fam132b | Fam132b | −4.07252 | 0.048462 | 1 |
| Fbxw18 | Fbxw18 | −4.07252 | 0.048462 | 1 |
| Fuom | Fuom | −4.07252 | 0.048462 | 1 |
| Gm5878 | Gm5878 | −4.07252 | 0.048462 | 1 |
| Oprl1 | Oprl1 | −4.07252 | 0.048462 | 1 |
| Slc6a13 | Slc6a13 | −4.07252 | 0.048462 | 1 |
| Eef1a2 | Eef1a2 | −1.60802 | 0.048748 | 0.804583 |
| Alkbh1 | Alkbh1 | −1.61313 | 0.04883 | 0.804583 |
| Ccdc175 | Ccdc175 | −4.07333 | 0.048882 | 1 |
| Clec4g | Clec4g | −4.07333 | 0.048882 | 1 |
| Kazald1 | Kazald1 | −4.07333 | 0.048882 | 1 |
| Ttbk1 | Ttbk1 | −4.07333 | 0.048882 | 1 |
| Snora44 | Snora44 | 1.758293 | 0.048891 | 0.804583 |
| Acot6 | Acot6 | −3.01994 | 0.048913 | 1 |
| Ralgds | Ralgds | 1.221898 | 0.048986 | 0.804583 |
| Naa35 | Naa35 | −1.26159 | 0.049347 | 0.804583 |
| Gipc1 | Gipc1 | 1.541166 | 0.049387 | 0.804583 |
| Ugt1a2 | Ugt1a2 | 1.165772 | 0.049424 | 0.804583 |
| Ugt1a5 | Ugt1a5 | 1.165772 | 0.049424 | 0.804583 |
| Ugt1a9 | Ugt1a9 | 1.165772 | 0.049424 | 0.804583 |
| Fcer2a | Fcer2a | −3.50917 | 0.049444 | 1 |
| Gp49a | Gp49a | 2.849744 | 0.049503 | 1 |
| Fiz1 | Fiz1 | −1.32831 | 0.049551 | 0.804583 |
| Angptl7 | Angptl7 | −4.07479 | 0.049663 | 1 |
| Anks1b | Anks1b | −4.07479 | 0.049663 | 1 |
| Edn2 | Edn2 | −4.07479 | 0.049663 | 1 |
| Fank1 | Fank1 | −4.07479 | 0.049663 | 1 |
| Fgf6 | Fgf6 | −4.07479 | 0.049663 | 1 |
| Gm13032 | Gm13032 | −4.07479 | 0.049663 | 1 |
| Kbtbd8 | Kbtbd8 | −4.07479 | 0.049663 | 1 |
| Kcnq2 | Kcnq2 | −4.07479 | 0.049663 | 1 |
| Mrgprx2 | Mrgprx2 | −4.07479 | 0.049663 | 1 |
| Fv1 | Fv1 | −2.75313 | 0.049807 | 1 |
| Rab1b | Rab1b | 1.008347 | 0.049831 | 0.804583 |
| Rasip1 | Rasip1 | 2.065895 | 0.049874 | 0.804583 |
| Alox12b | Alox12b | 3.086733 | 0.049896 | 1 |

Differences in Extracellular Vesicles RNA-Content as a Function of Age

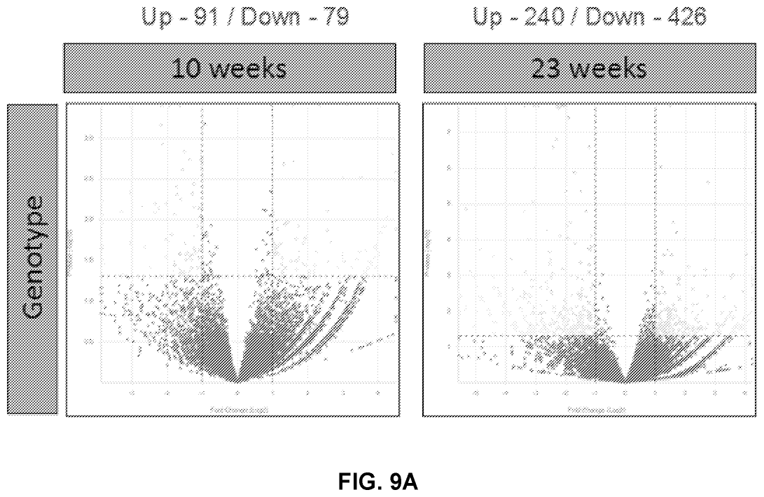

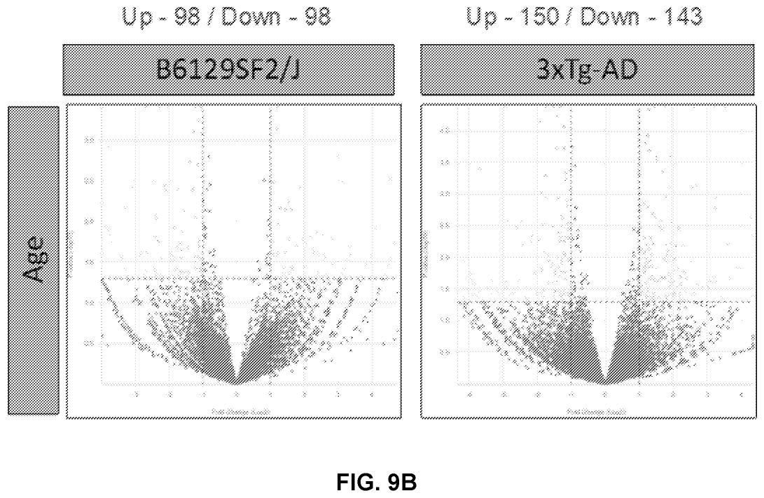

Differential expression and IPA analyses were performed on 3×Tg-AD 23 weeks vs. 3×Tg-AD 10 weeks and B6129SF2/J 23 weeks vs. B6129SF2/J 10 weeks to determine the effect that age has in both the AD and control mice. A total of 150 upregulated genes and 143 downregulated genes were found in 3×Tg-AD 23 week vs. 3×Tg-AD 10-week comparison (FIG. 9B). Pathway analysis of these genes revealed the dysregulation of several pathways, including hepatic fibrosis, GP6 signaling, and apelin liver signaling. The comparison of B6129SF2/J 23 weeks vs. B6129SF2/J 10 weeks was performed to evaluate the changes in expression due to aging alone. The results revealed the upregulation of 98 genes, as well as the downregulation of 98 others (Table 2). Top related pathways that were identified include unfolded protein response, RhoA Signaling, and adrenomedullin signaling (FIG. 10A, 10B).

TABLE 2

| Name | Description | log2FoldChange | pValue | Padj |
|---|---|---|---|---|
| Scap | Scap | −2.67827 | 2.71E−16 | 3.23E−13 |
| Vaultrc5 | Vaultrc5 | −2.01653 | 2.16E−09 | 1.29E−06 |
| Serpina3j | Serpina3j | −2.02098 | 8.04E−07 | 0.00032 |
| Rps3a1 | Rps3a1 | −1.22472 | 5.22E−06 | 0.001559 |
| Ttn | Ttn | 1.213967 | 3.13E−05 | 0.0074 |
| Elovl4 | Elovl4 | −1.05938 | 3.72E−05 | 0.0074 |
| Psapl1 | Psapl1 | −1.04776 | 0.000358 | 0.057057 |
| Ces1d | Ces1d | −2.31005 | 0.000382 | 0.057057 |
| S100a9 | S100a9 | −5.66722 | 0.0004 | 1 |
| Rnase2b | Rnase2b | −5.61867 | 0.000444 | 1 |
| Mcl1 | Mcl1 | −1.14158 | 0.000569 | 0.075544 |
| Tpm1 | Tpm1 | 1.200706 | 0.000719 | 0.081237 |
| Dhcr24 | Dhcr24 | −1.26664 | 0.00078 | 0.081237 |
| Ctsd | Ctsd | −1.01856 | 0.000816 | 0.081237 |
| Cxcl2 | Cxcl2 | −3.79001 | 0.001196 | 1 |
| Akr1c18 | Akr1c18 | 2.999493 | 0.001585 | 1 |
| Snord22 | Snord22 | −1.38341 | 0.001834 | 0.156386 |
| Serpina3b | Serpina3b | −1.14557 | 0.001992 | 0.158581 |
| Rgs9 | Rgs9 | −1.0233 | 0.002814 | 0.20553 |
| Hist1h2ab | Hist1h2ab | 4.551693 | 0.002968 | 1 |
| Acta1 | Acta1 | 1.002455 | 0.003051 | 0.20553 |
| Lphn3 | Lphn3 | 5.126657 | 0.003063 | 1 |
| Il1b | Il1b | −2.46363 | 0.003211 | 1 |
| Neb | Neb | 1.090694 | 0.003271 | 0.20553 |
| Jph2 | Jph2 | 3.144739 | 0.003293 | 1 |
| Fbxo40 | Fbxo40 | 5.082152 | 0.003565 | 1 |
| Iigp1 | Iigp1 | −2.11099 | 0.003597 | 1 |
| Gse1 | Gse1 | −3.10302 | 0.003768 | 1 |
| Prpf40a | Prpf40a | 1.883555 | 0.003801 | 1 |
| Ly6g6c | Ly6g6c | −1.29149 | 0.003945 | 0.206967 |
| Irf1 | Irf1 | −1.73793 | 0.004124 | 0.206967 |
| Gbp2 | Gbp2 | −2.09345 | 0.004148 | 1 |
| Slc38a2 | Slc38a2 | −1.31748 | 0.00416 | 0.206967 |
| Anxa3 | Anxa3 | 1.53754 | 0.005685 | 0.261053 |
| Dbp | Dbp | −4.83447 | 0.0059 | 1 |
| S100a8 | S100a8 | −3.29959 | 0.006723 | 1 |
| Lars2 | Lars2 | 1.290893 | 0.006765 | 0.295033 |
| Cux1 | Cux1 | 1.629415 | 0.007082 | 1 |
| Zfp560 | Zfp560 | 4.920765 | 0.007108 | 1 |
| Actn3 | Actn3 | 1.448642 | 0.007158 | 0.295033 |
| Ces4a | Ces4a | −1.5667 | 0.007347 | 1 |
| Mitf | Mitf | −2.3857 | 0.0085 | 1 |
| Tgoln1 | Tgoln1 | −1.20235 | 0.008864 | 0.341407 |
| Srgn | Srgn | −2.36225 | 0.009253 | 1 |
| Mylpf | Mylpf | 1.232371 | 0.010863 | 0.376635 |
| Ryr1 | Ryr1 | 1.229883 | 0.010966 | 0.376635 |
| Igfn1 | Igfn1 | 4.794133 | 0.011266 | 1 |
| 2310002L09Rik | 2310002L09Rik | 4.818736 | 0.011423 | 1 |
| Xaf1 | Xaf1 | −3.87613 | 0.011565 | 1 |
| Hmox1 | Hmox1 | −1.85346 | 0.012013 | 1 |
| Col4a4 | Col4a4 | 4.129727 | 0.01213 | 1 |
| Mapk13 | Mapk13 | −3.79302 | 0.01226 | 1 |
| B3glct | B3glct | 4.139149 | 0.012501 | 1 |
| Mroh2a | Mroh2a | 4.772877 | 0.01252 | 1 |
| Hcar2 | Hcar2 | −3.79597 | 0.012641 | 1 |
| Elovl3 | Elovl3 | −1.60337 | 0.013158 | 1 |
| Ggta1 | Ggta1 | 2.387077 | 0.013822 | 1 |
| Scarna2 | Scarna2 | −3.73829 | 0.014679 | 1 |
| E030003E18Rik | E030003E18Rik | 2.545239 | 0.014861 | 1 |
| Mxd1 | Mxd1 | −2.41606 | 0.015368 | 1 |
| Mb | Mb | 2.193699 | 0.0154 | 1 |
| Myot | Myot | 1.983929 | 0.015701 | 1 |
| Smtnl1 | Smtnl1 | 2.843728 | 0.016347 | 1 |
| Trdn | Trdn | 1.960649 | 0.016729 | 1 |
| Myh2 | Myh2 | 1.546649 | 0.016762 | 1 |
| Pnpla2 | Pnpla2 | −1.11759 | 0.01715 | 0.472193 |
| Clca2 | Clca2 | −2.32086 | 0.017401 | 0.472193 |
| Saa3 | Saa3 | −3.69364 | 0.017505 | 1 |
| Baiap3 | Baiap3 | 4.679702 | 0.017658 | 1 |
| Prickle3 | Prickle3 | 4.679702 | 0.017658 | 1 |
| Mrgprg | Mrgprg | −4.37746 | 0.017778 | 1 |
| Crip2 | Crip2 | 1.08262 | 0.017862 | 0.473947 |
| Kprp | Kprp | 2.037065 | 0.017974 | 1 |
| H2-T23 | H2-T23 | −1.67094 | 0.018654 | 1 |
| Cmss1 | Cmss1 | 4.71226 | 0.019045 | 1 |
| Cdr1 | Cdr1 | 4.654431 | 0.019229 | 1 |
| Smad1 | Smad1 | 1.777526 | 0.019968 | 1 |
| Klf9 | Klf9 | −1.11282 | 0.02014 | 0.52276 |
| L2hgdh | L2hgdh | 3.342868 | 0.020452 | 1 |
| Nr1d1 | Nr1d1 | −1.76272 | 0.020713 | 1 |
| Manf | Manf | −1.44295 | 0.021053 | 1 |
| Fmod | Fmod | 2.260675 | 0.021854 | 1 |
| Fam102a | Fam102a | −1.63857 | 0.022495 | 1 |
| Mcpt4 | Mcpt4 | −1.61799 | 0.022791 | 1 |
| Polq | Polq | 4.627446 | 0.023 | 1 |
| Ndufv2 | Ndufv2 | 1.442308 | 0.02386 | 1 |
| Glb1l2 | Glb1l2 | −2.65042 | 0.024039 | 1 |
| Rnu11 | Rnu11 | 2.291792 | 0.024268 | 0.568562 |
| Cxcr2 | Cxcr2 | −4.27521 | 0.024309 | 1 |
| Ccl4 | Ccl4 | −4.31817 | 0.024546 | 1 |
| F13a1 | F13a1 | 1.217863 | 0.024855 | 0.568562 |
| Nr1d2 | Nr1d2 | −1.96682 | 0.024947 | 1 |
| Sfi1 | Sfi1 | 2.42208 | 0.025023 | 1 |
| Paqr7 | Paqr7 | −2.84995 | 0.025082 | 1 |
| Cilp | Cilp | 1.092303 | 0.025523 | 0.568562 |
| Akr1e1 | Akr1e1 | 3.384689 | 0.025574 | 1 |
| Hephl1 | Hephl1 | 2.581716 | 0.02563 | 1 |
| Cmya5 | Cmya5 | 1.211069 | 0.026063 | 0.568562 |
| Cma1 | Cma1 | −1.88069 | 0.026187 | 1 |
| Cdsn | Cdsn | −1.15008 | 0.026806 | 0.571551 |
| Tsku | Tsku | −3.42553 | 0.026952 | 1 |
| Myh7 | Myh7 | 3.971758 | 0.027014 | 1 |
| Tgoln2 | Tgoln2 | −1.23471 | 0.027106 | 1 |
| Col24a1 | Col24a1 | 4.556997 | 0.027867 | 1 |
| Nhlrc1 | Nhlrc1 | 4.556997 | 0.027867 | 1 |
| Fcna | Fcna | 2.628556 | 0.027903 | 1 |
| Icam1 | Icam1 | −1.64038 | 0.028018 | 1 |
| Samhd1 | Samhd1 | −1.08289 | 0.028341 | 0.583426 |
| Zfp131 | Zfp131 | −2.21832 | 0.028416 | 1 |
| Kctd5 | Kctd5 | −3.38545 | 0.028866 | 1 |
| Cdk14 | Cdk14 | −1.83338 | 0.029275 | 1 |
| Tcea3 | Tcea3 | 3.284257 | 0.029472 | 1 |
| Cntn2 | Cntn2 | 4.526251 | 0.029497 | 1 |
| Hydin | Hydin | 4.526251 | 0.029497 | 1 |
| Tmppe | Tmppe | −4.1694 | 0.029653 | 1 |
| Ctgf | Ctgf | −1.83646 | 0.029903 | 1 |
| Rbm20 | Rbm20 | 4.59375 | 0.030221 | 1 |
| Utp23 | Utp23 | 4.59375 | 0.030221 | 1 |
| Smpd3 | Smpd3 | −1.98743 | 0.03025 | 1 |
| Trp53inp1 | Trp53inp1 | −1.89828 | 0.030315 | 1 |
| Alkbh1 | Alkbh1 | 3.22294 | 0.030506 | 1 |
| Cstf1 | Cstf1 | 2.59779 | 0.030868 | 1 |
| Ephx2 | Ephx2 | 2.894185 | 0.031344 | 1 |
| Al607873 | Al607873 | −1.21839 | 0.031415 | 0.604747 |
| Spta1 | Spta1 | 3.837058 | 0.032429 | 1 |
| Sspo | Sspo | 3.82196 | 0.033066 | 1 |
| Zfp799 | Zfp799 | 3.822092 | 0.033167 | 1 |
| Asb2 | Asb2 | 2.839846 | 0.033242 | 1 |
| Cys1 | Cys1 | −4.12111 | 0.033348 | 1 |
| Slfn9 | Slfn9 | 4.495341 | 0.034601 | 1 |
| Zxdb | Zxdb | 4.495341 | 0.034601 | 1 |
| Trmt10c | Trmt10c | 3.808102 | 0.034838 | 1 |
| Wdr35 | Wdr35 | 3.808141 | 0.034955 | 1 |
| Alpk3 | Alpk3 | 2.038776 | 0.036105 | 1 |
| Tnnc2 | Tnnc2 | 1.632351 | 0.036761 | 0.622832 |
| Htt | Htt | 1.396217 | 0.037036 | 1 |
| Tbc1d2b | Tbc1d2b | −1.19107 | 0.037599 | 0.622832 |
| Ctdspl | Ctdspl | −1.77535 | 0.038098 | 1 |
| Prdx6 | Prdx6 | −1.06182 | 0.038101 | 0.622832 |
| Tcf7l1 | Tcf7l1 | −2.42812 | 0.03814 | 1 |
| Ggps1 | Ggps1 | 3.184304 | 0.038718 | 1 |
| Rgs5 | Rgs5 | 1.653026 | 0.038888 | 1 |
| Gdpd3 | Gdpd3 | −3.32327 | 0.039262 | 1 |
| Fbxo30 | Fbxo30 | −1.82562 | 0.039641 | 1 |
| Snora70 | Snora70 | −3.3071 | 0.039848 | 1 |
| Gm13177 | Gm13177 | −2.66533 | 0.039862 | 1 |
| Cyp2e1 | Cyp2e1 | −1.68632 | 0.040206 | 1 |
| Galnt15 | Galnt15 | −1.07027 | 0.040324 | 0.622832 |
| Gbp7 | Gbp7 | −1.65182 | 0.041145 | 1 |
| Etv3 | Etv3 | −1.45129 | 0.041283 | 1 |
| Tshz1 | Tshz1 | −2.40036 | 0.04149 | 1 |
| Dennd2a | Dennd2a | −1.66301 | 0.04211 | 1 |
| Ubl3 | Ubl3 | −1.41248 | 0.042457 | 1 |
| Kat2b | Kat2b | −1.56039 | 0.042667 | 1 |
| Nup133 | Nup133 | 2.430995 | 0.04287 | 1 |
| Thbs4 | Thbs4 | 1.569851 | 0.04364 | 1 |
| Pld4 | Pld4 | 3.13155 | 0.043899 | 1 |
| Agpat6 | Agpat6 | 1.413953 | 0.044267 | 1 |
| Fa2h | Fa2h | −1.01459 | 0.044513 | 0.664364 |
| Pecam1 | Pecam1 | −1.72964 | 0.044783 | 1 |
| Cpne7 | Cpne7 | 4.42578 | 0.044784 | 1 |
| Gspt2 | Gspt2 | 4.42578 | 0.044784 | 1 |
| Ildr1 | Ildr1 | 4.42578 | 0.044784 | 1 |
| Krt83 | Krt83 | 4.42578 | 0.044784 | 1 |
| Lrrc39 | Lrrc39 | 4.42578 | 0.044784 | 1 |
| Syn1 | Syn1 | 4.42578 | 0.044784 | 1 |
| Fbxl15 | Fbxl15 | 4.386558 | 0.045002 | 1 |
| Neurl1a | Neurl1a | 4.386558 | 0.045002 | 1 |
| Pla2g4f | Pla2g4f | 4.386558 | 0.045002 | 1 |
| Slc38a3 | Slc38a3 | 4.386558 | 0.045002 | 1 |
| Cd53 | Cd53 | −2.75177 | 0.045188 | 1 |
| Gcnt2 | Gcnt2 | −2.37191 | 0.04522 | 1 |
| Nrbp2 | Nrbp2 | 2.388124 | 0.045347 | 1 |
| Gskip | Gskip | 2.647855 | 0.045352 | 1 |
| Ubc | Ubc | −1.91332 | 0.045826 | 1 |
| Nup37 | Nup37 | 3.70438 | 0.046102 | 1 |
| Sema6b | Sema6b | 3.70438 | 0.046102 | 1 |
| B4galt2 | B4galt2 | −3.22448 | 0.046208 | 1 |
| Gna12 | Gna12 | −2.251 | 0.046532 | 1 |
| Dpep1 | Dpep1 | −1.01581 | 0.046821 | 0.664666 |
| Pcsk7 | Pcsk7 | 2.468747 | 0.047038 | 1 |
| Wdr60 | Wdr60 | 2.702734 | 0.047617 | 1 |
| Atp6v1b2 | Atp6v1b2 | −1.45926 | 0.047619 | 1 |
| Golga3 | Golga3 | 1.242583 | 0.047926 | 1 |
| Tchh | Tchh | 2.327368 | 0.048297 | 1 |
| Psmb9 | Psmb9 | −2.56932 | 0.048438 | 1 |
| Sos2 | Sos2 | −2.13253 | 0.049009 | 1 |
| Sertad1 | Sertad1 | −1.63071 | 0.049113 | 1 |
| Ifi47 | Ifi47 | −2.26909 | 0.049224 | 1 |
| Senp7 | Senp7 | 2.652941 | 0.049255 | 1 |
| Irgm2 | Irgm2 | −1.71911 | 0.049343 | 1 |
| Meg3 | Meg3 | 3.731533 | 0.049397 | 1 |
| Wdr37 | Wdr37 | 2.163074 | 0.049697 | 1 |
| Serpina3g | Serpina3g | −2.74284 | 0.049711 | 1 |
| Hook1 | Hook1 | 2.654264 | 0.049802 | 1 |
| Gng2 | Gng2 | −2.70517 | 0.049842 | 1 |

Differences in Extracellular Vesicles RNA-Content as an Interaction Between Age and Genotype:

Two final sets of differential expression analysis were performed, 3×Tg-AD 10 weeks vs. B6129SF2/J 23 weeks and 3×Tg-AD 23 weeks vs. B6129SF2/J 10 weeks. These comparisons were made in order to evaluate the effect of both age and genotype on the mRNA expression profile in skin-derived EVs. The 3×Tg-AD 10 weeks vs. B6129SF2/J 23 weeks analysis indicated significant upregulation of 67 genes and the downregulation of 89 genes, with no genes being upregulated. Several top pathways that were implicated with these genes include calcium signaling, actin cytoskeleton signaling, and calcium transport I. Analysis of the 3×Tg-AD 23 weeks vs. B6129SF2/J 10 weeks comparison revealed the upregulation of 122 genes and downregulation of 134 genes. Subsequent pathway analysis implicated GP6 signaling, corticotropin releasing hormone, and hepatic fibrosis pathways as related to these differentially expressed genes.

Murine model 3×tgAD Skin-Derived Extracellular Vesicles Contain Transgenic hAPP/hMAPT mRNA

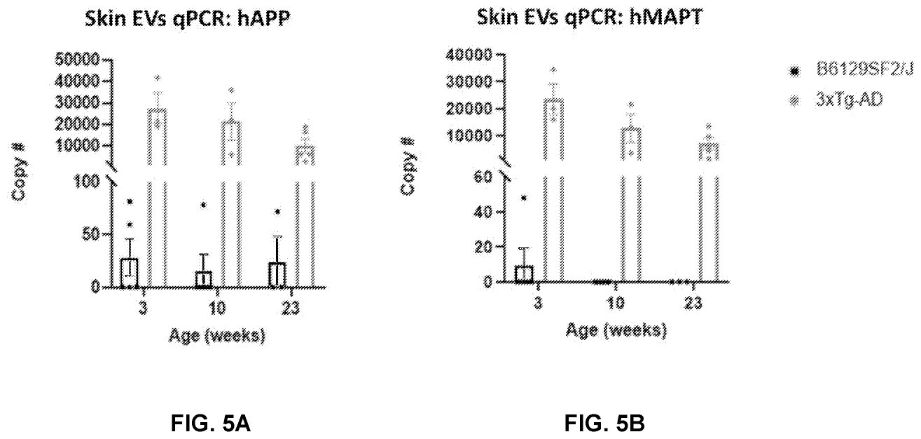

Mutations and aberrant expression of genes related to APP are well documented factors involved in the propagation of neurodegenerative disorders involving amyloidosis such as AD (Shin, J., et al. BMB Rep. 2010 43:704-709; Strang, K H., et al. Lab. Invest. 2019 99:912-928). While mutations in microtubule associated protein Tau (MAPT) are not directly associated with AD (Guo, Q. et al. PLoS One 2013 8:e80706), they have been implicated in frontotemporal dementia (Strang, K H., et al. Lab. Invest. 2019 99:912-928; Guo, Q. et al. PLoS One 2013 8:e80706). More importantly however, these mutations are responsible for the development of tauopathy similar to that seen in AD and can be found in transgenic models of AD (Guo, Q. et al. PLoS One 2013 8:e80706). Here the potential for skin-derived EVs collected was evaluated from both 3×Tg-AD and B6129SF2/J control mice to contain human APP Swedish Mutation (APPswe) and human Tau P301L MAPT transgenes expressed in the 3×Tg-AD murine model of Alzheimer's Disease (Oddo, S. et al. Neuron 2003 39:409-421). The presence of these transgenes were characterized through the use of absolute qPCR (n=3) to measure the expression of hAPP and hMAPT mRNA in skin-derived EVs collected at various time points (FIG. 5). The resulting gene expression data shows that both hAPP (FIG. 5A) and hMAPT (FIG. 5B) are expressed significantly in 3×Tg-AD skin-derived EVs compared to age matched controls, further indicating that they are capable of harboring potentially neurotoxic genetic cues in relation to the progression of AD within this model.

3×Tg-AD Skin-Derived Extracellular Vesicles can Transfer Neurotoxic hAPP and hMAPT mRNA to Neurons in Murine Primary Embryonic Neuron Cultures

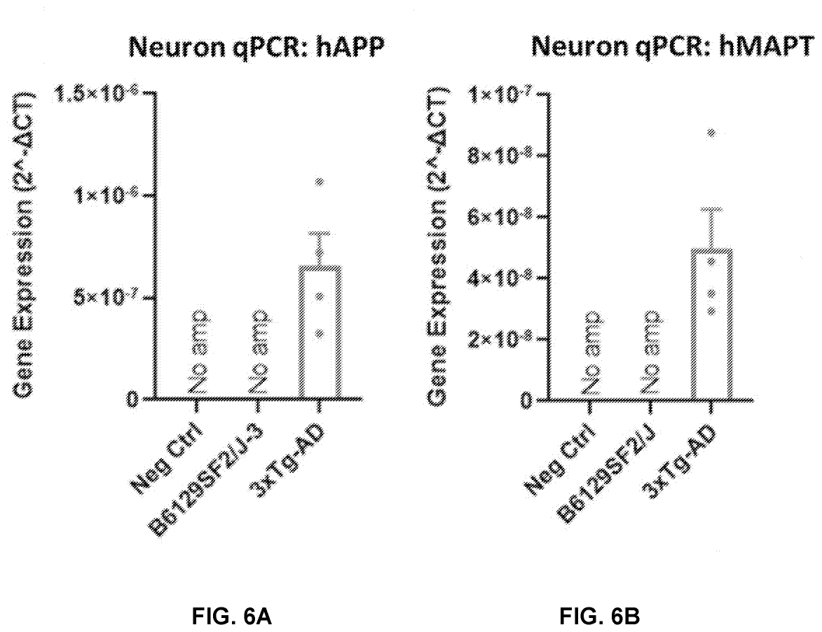

Based on results indicating that 3×Tg-AD skin-derived EVs are capable of harboring potentially neurotoxic molecular contents, it was decided to proceed and evaluate the neurotoxic capabilities of these EVs directly. While EVs derived from the central nervous system have previously been shown to play a role in neurodegenerative disorders such as Alzheimer's Disease (Sardar Sinha, M. et al. Acta Neuropathol. 2018 136:41-56) and that exosomal proteins are found within amyloid plaques (Rajendran, L. et al. Proc. Natl. Acad. Sci. U.S.A 2006 103:11172-11177), this pathological potential has yet to be explored in regards to EVs derived from peripheral tissues such as the skin. Therefore, the neurotoxic capabilities of 3×Tg-AD and B6129SF2/J skin-derived EVs were examined by exposing 4 different cultures of murine primary neuron cultures to EVs at a concentration of 1-3×10⁹ EVs/μL particles for 24 hours followed by RNA isolation for qPCR analysis. The subsequent qPCR results (n=4) show that transgenic hAPP and hMAPT mRNA is significantly expressed in neurons exposed to 3×Tg-AD EVs compared to controls (FIG. 6A, 6B). These findings indicate that 3×Tg-AD skin-derived EVs have the potential to influence the genetic expression profile of neuron targets through the delivery of transgenic mRNA. This observed activity could possibly lead to neurotoxic effects due to expression of APPswe and P301L Tau mutant transgenes, which could in turn advance the onset and/or progression of AD.

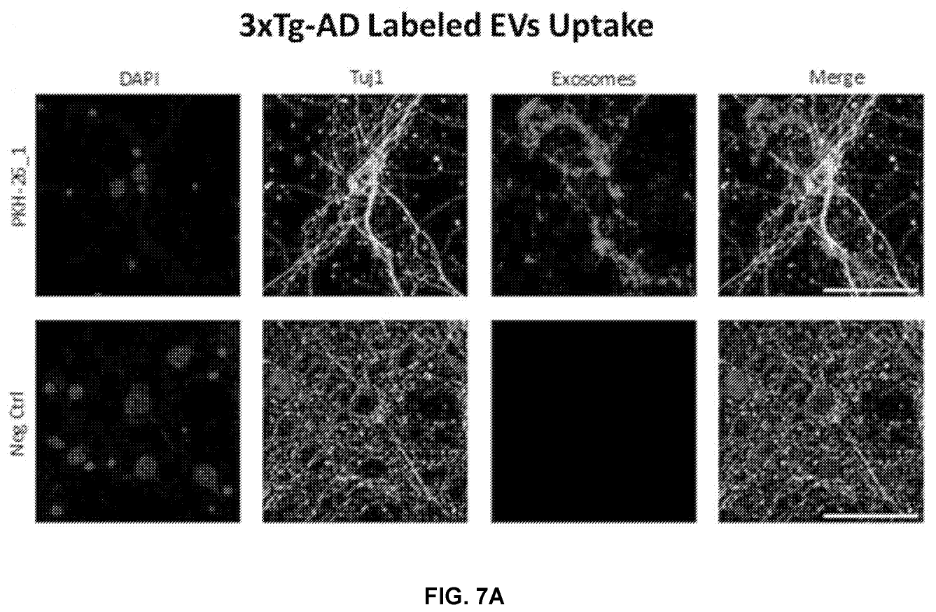

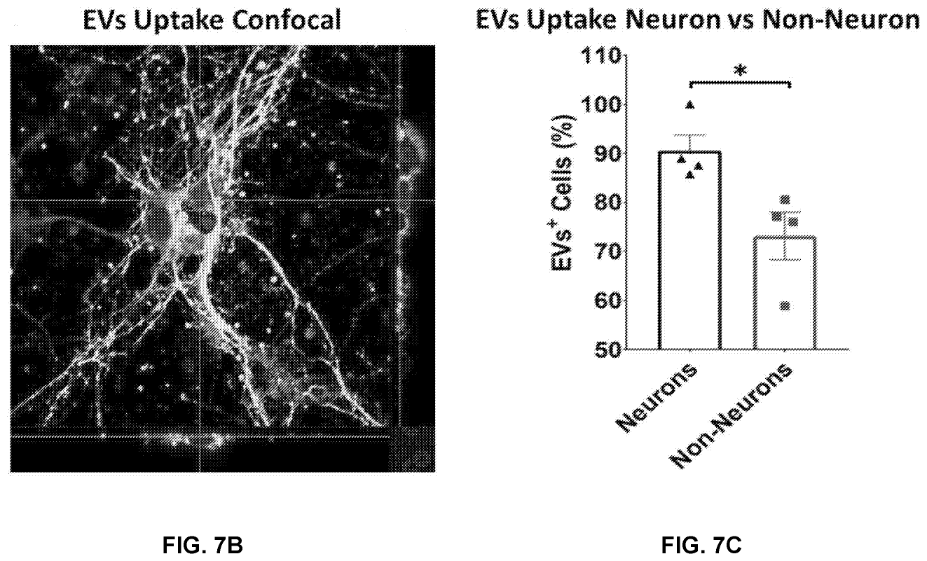

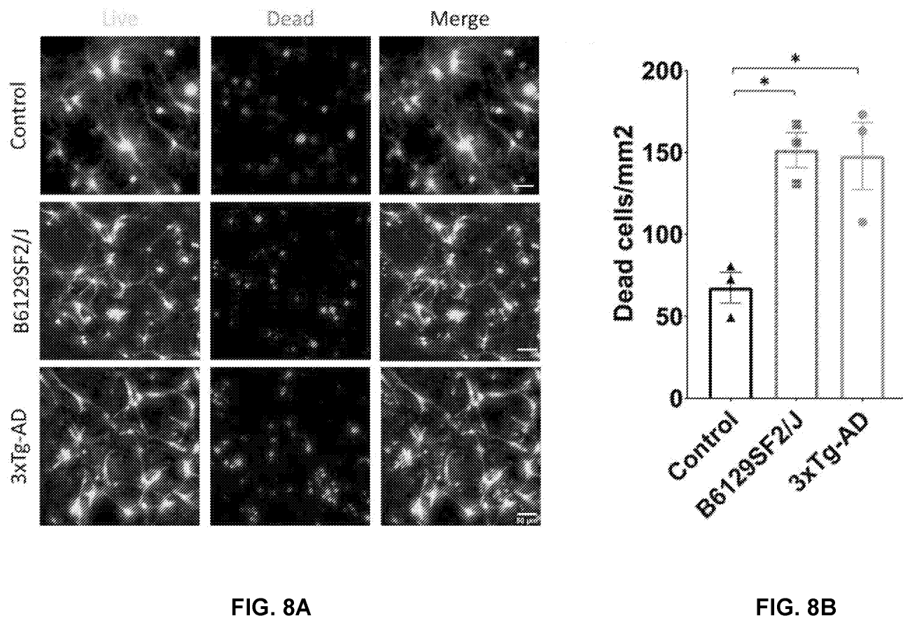

Next the effects of skin-derived EVs on primary neuronal cultures, was evaluated. Equivalent amounts of skin-derived EVs isolated from B612SF2/J and 3×Tg-AD at 10 weeks of age. First, EVs from each experimental group were labeled with the PKH26 (Sigma) red fluorescent label and then applied to 8-days primary neuronal cultures for 24h (FIG. 7). Qualitative analysis by confocal microscopy shows the presence of fluorescent particles in the cytoplasm of neurons, including both the perinuclear area and the neuronal projections, identified by Tuj1 immunostaining (FIG. 7A, 7B). Furthermore, quantification of cellular uptake shows a higher percentage of neurons uptaking the fluorescent-labeled EVs (FIG. 7C). Finally, the neurotoxic effects of EVs derived from the AD mouse model (FIG. 8) was studied. 24 hours after exposing primary neuronal cultures to 3×Tg-AD or B6129SF2/J skin-derived EVs cell viability was evaluated by epifluorescence microscopy using the Live/Dead kit (FIG. 8A). Quantification of neuronal viability shows a cytotoxic effect of EVs in general when compared with unexposed control cultures, however, after 24h there is no difference in cell toxicity between EVs derived from B6129SF2/J or 3×Tg-AD mice (FIG. 8B).

Discussion

The role that peripheral organs and tissues such as the skin and gut play in the onset and progression of neurodegenerative disorders, such as AD and PD, has only recently begun to be understood. While the skin has been suspected to play a role in AD specifically and has even been explored as a possible avenue of diagnosis and drug delivery, no particular mechanism detailing how such a link might be facilitated has been identified. Through the results of the experiments aimed at characterizing the genetic content of skin-derived EVs as AD progresses, changes were identified in the expression of AD related genes such as S100A9 (FIG. 4A), as well as expression of APPswe and MAPT P301L mutant genes (FIG. 5) responsible for driving the pathology in the 3×Tg-AD model of AD.

Calcium signaling was among the top dysregulated canonical pathways identified by IPA among all the comparison groups. This observation was found in the comparison of differentially expressed genes among the 3×Tg-AD 23 week and B6129SF2/J 23 week groups, but absent in the comparison of the 3×Tg-AD 10 week and B6129SF2/J 10 week groups. This observation suggests that changes in mRNA expression involved in calcium signaling occur with age in the 3×Tg-AD mice that are not seen in B6129SF2/J controls. The dysregulation of calcium signaling has been documented in neurodegenerative diseases such as AD for over a decade and has led to the development of the “calcium hypothesis” of AD (Tong, B C K., et al. Biochim. Biophys. Acta Mol. Cell Res. 2018 1865:1745-1760; Popugaeva, E., et al. Antioxid. Redox Signal. 2018 29:1176-1188). There is growing evidence that the dysregulation of calcium homeostasis in neurons causes synaptic deficits that ultimately lead to the accumulation of amyloid-beta and phosphorylated tau 29 (Popugaeva, E., et al. Antioxid. Redox Signal. 2018 29:1176-1188). In addition to disruptions in calcium signaling, mitochondrial dysfunction, specifically associated with mitochondria-associated ER membrane (MAM), has also been reported in AD (Chakravorty, A., et al. Front. Aging Neurosci. 2019 11:311). While not identified through IPA as a canonical pathway, two genes involved with calcium signaling within the 3×Tg-AD 23 week vs B6129SF2/J 23 week group, ATP2A1 and RYR1, are also associated with MAM function (Schon, E A. & Area-Gomez, E. et al. Mol. Cell. Neurosci. 2013 55:26-36). While MAM associated genes are generally upregulated in AD, the perturbation of MAM function overall is a suspected component of the AD pathology and represents its own hypothesis for how the disease is caused (Area-Gomez, E. & Schon, E A. et al. Curr. Opin. Genet. Dev. 2016 38:90-96).

Another intriguing find within the 3×Tg-AD and B6129SF2/J 23 week group comparisons was the robust upregulation of S100A9 (Log 2FoldChange=6.155). S100A9 is a member of the S100 protein family, which is known to be involved in a multitude of intracellular processes including calcium homeostasis (Cristóvão, J S. & Gomes, C M. SNeurosci. 2019 13:463). As mentioned before, observed differences among the groups in this comparison provide insight into changes in gene expression that occur in skin EVs as AD progression occurs. As such, the finding that S100A9 is heavily upregulated suggests this could be a key change in the genetic profile of skin EVs as AD progresses. Interestingly, this expression pattern is consistent with changes seen in the brain of related TG2576 AD mouse models (Cristóvão, J S. & Gomes, C M. SNeurosci. 2019 13:463). This finding in TG2576 mice is particularly relevant, as this model bears the same APPswe mutations found in the 3×Tg-AD used in this study. Furthermore, studies involving the use of S100A9 KO bred with TG2576 mice showed improvements in memory impairment and decreased neuropathology (Kim, H. J. et al. PLoS One 2014 9:e88924). In addition to murine models, upregulation of S100A9 expression has been observed in the brains of AD patients and implicated in AD pathways (Cristóvão, J S. & Gomes, C M. SNeurosci. 2019 13:463). For these reasons, S100A9 has gained a lot of attention as a potential biomarker of AD (Horvath, I. et al. ACS Chem. Neurosci. 2016 7:34-39). Further exploration of the changes and effects of both S100A9 mRNA and protein within skin-derived EVs under AD conditions could prove to be insightful for such purposes. The upregulation of sodium-coupled neutral amino acid transporter 2 (SLC38A2) was also observed within this comparison group. The upregulation of SLC38A2 in 23 week 3×Tg-AD skin-derived EVs is consistent with observations of SLC38A2 upregulation with AD progression (Patel, H. et al. Brain Behav. Immun. 2019 80:644-656). The gene for Serpina3b/Serpina3j was found to be upregulated within the 3×Tg-AD and B6129SF2/J 23 week group comparisons as well. The complete opposite result was seen when in the B6129SF2/J 23 weeks vs. B6129SF2/J 10 weeks group, where Serpina3b/Serpina3j was found to be downregulated. The fact that this gene is heavily expressed in aged AD mice while declining with age in controls suggests that its expression is due to the AD condition. This observation of upregulation of Serpina3b/Serpina3j in the skin-derived EVs 3×Tg-AD mice is consistent with previous observations of serpina3 upregulation in multiple prion diseases, including AD (Vanni, S. et al. Sci. Rep. 2017 7:15637).

Absolute qPCR experiments aimed at measuring the expression of transgenic mRNA related to AD in skin-derived EVs further support the notion that they could play a role in AD. The finding that human APPswe and human MAPT P301L mRNA is present in AD skin-derived EVs supports the hypothesis that they are capable of driving the onset and progression of AD. This ultimately suggests the possibility that these EVs are capable of delivering their transgenic mRNA contents to target tissues, which can influence the local protein expression of mutated MAPT and APP within said tissue. This is particularly relevant to AD within the context of the 3×Tg-AD model, where the expression of APPswe and MAPT P301L within the CNS is a major component of the pathology.

In addition, to characterize the effects of mRNAs in AD skin-derived EVs, their neurotoxic potential was evaluated directly in primary neuron EVs exposure experiments. The ability of neurons to uptake mRNA of hAPP and hMAPT from AD skin EVs was demonstrated, suggesting the possibility that aberrant expression of mutated APP and MAPT proteins could possibly occur, which could drive disease progression through subsequent accumulations of beta-amyloid and phosphorylated Tau proteins in the CNS. The results of labeled EVs uptake experiments in primary neurons further support this idea by providing evidence that skin-derived EVs themselves are in fact capable of being uptaken by Tuj1⁺ cells (FIG. 7A, 7B) and do so preferentially over Tuj1⁻ cells in vitro (FIG. 7C). Taken together, these observations highlight the potential for skin-derived EVs to interact with neurons and influence expression of AD-related genes within the CNS, ultimately contributing to disease onset and progression. Finally, despite the transfer of potentially neurotoxic cargo from skin-EVs of the 3×Tg-AD mice observed here by qRT-PCR and cellular uptake experiments, no increase in neuron toxicity was observed after exposure to 3×Tg-AD skin EVs, this may be due to the fact a short-term exposure (24h) was performed (FIG. 8), and the cytotoxic effects of APP and Tau may require further incubation times to allow metabolic processing, oligomer formation, and protein accumulation.

Methods

Animal Studies

Breeding trios of the triple transgenic mouse model of Alzheimer's Disease (3×Tg-AD) B6; 129-Tg(APPSwe,TauP301L)1Lfa Psen1^{tm1Mpm}/Mmjax (Mutant Mouse Resource & Research Centers Stock No: 34830-JAX) (Oddo, S. et al. Neuron 2003 39:409-421) and the control strain B6129SF2/J (Stock No: 101045) were purchased from The Jackson Laboratory. Based on recommendations from the donating investigator, only female animals were used for analysis. Mice were housed in groups with ad libitum access to water and food in a 12/12 h light/dark cycle (lights on at 6 A.M.) with constant temperature and humidity conditions. All experiments involving animals were performed in accordance with The Ohio State University Institutional Animal Care and Use Committee guidelines (protocol number: 2016A00000074-R1).

Skin Extracellular Vesicles Isolation

Murine skin-derived EVs were isolated directly from dorsal and ventral 12-mm-diameter skin biopsies. The collected tissue was minced into −1 mm pieces with a surgical scalpel and dissociated using the “37_Multi H” protocol on a “gentleMACS Octo Dissociator” (Miltenyi Biotec Cat. No. 130-096-427) in combination with Multi Tissue Dissociation Kit 1 (Miltenyi Biotec #130-110-201). The resulting supernatant was then removed centrifuged at 2000g at 4° C. for 30 minutes. The supernatant was then removed and pooled to ensure homogenous EVs concentration and then 1/2 volume of “Total Exosome Isolation Kit from Cells” (Thermo Fisher Scientific #4478359) was added to it prior to a 12 hour incubation at 4° C. The EVs samples were precipitated with a 10,000g centrifugation at 4 degrees and stored afterwards at −80° C. until future use. EVs concentration was measured using a NanoSight Ns3000 (Malvern Pananlytical).

Absolute Real Time PCR of Skin Extracellular-Vesicles

Total RNA was extracted from skin EVs pellets using the TRIzol reagent (Thermo Fisher Scientific #15596026) following manufacturer's instructions, followed by measurement of resulting concentration using a NanoDrop 2000 Spectrophotometer (Thermo Fisher #ND-2000). The VILO cDNA synthesis kit (Thermo Fisher Scientific #11756500) was used to perform 20 uL reverse transcription reactions. The resulting cDNA was used to measure expression of hAPP and hMAPT using absolute quantification methods with standard curves of plasmids for APP695 (Addgene Plasmid #114193) and TauP301L (Addgene Plasmid #87633) that were generated with a final copy number range of 10\hat{ }6 copies to 10\hat{ }2 copies. The aforementioned plasmids were isolated using ZymoPURE II Plasmid Midi Prep Kit (Zymo Research #DG4200). Measurement of isolated plasmid concentration was conducted using a NanoDrop 2000 Spectrophotometer (Thermo Fisher #ND-2000), followed by subsequent calculation of plasmid copy number and dilution of the standard curve. Taqman primers from ThermoFisher were used to amplify genes of interest including: hAPP (Thermo Fisher Scientific #Hs00169098_m1), mAPP (Thermo Fisher Scientific #Hs00169098_m1), hMAPT (Thermo Fisher Scientific #Hs00902194_m1), and mMAPT (Thermo Fisher Scientific #Mm00521988_m1). All real-time PCR reactions were performed using TaqMan Fast Advanced Master Mix (Thermo Fisher Scientific #44-445-57) and a QuantStudio 3 Real-Time PCR System (Thermo Fisher Scientific #A28567) using the following cycle settings: 95° C. for 10 minutes, followed by 40 cycles of 95° C. for 1 minute, 60° C. for 1 minute, and 72° C. for 1 minute.

Relative Real Time PCR of Primary Neurons

RNA isolation and cDNA generation were performed using the aforementioned techniques and reagents. Real Time PCR was subsequently performed on primary neurons using the previously mentioned reagents, primers, thermo cycler, and cycle settings. Mouse 18s Ribosomal RNA (Thermo Fisher Scientific #4331182) was used as a housekeeping gene.

RNA-Seq

Total RNA was extracted from extracellular vesicles (EVs) derived from the skin of B6129SF2/J or 3×Tg-AD mice using TRIzol™ reagent (ThermoFisher Scientific #15596026) following the manufacturer's directions as previously described. RNA quantification, integrity and quality assessment was obtained with the Qubit Fluorometer (ThermoFisher Scientific #15596026). Libraries were generated with an input of 200 ng per sample using the NEBNext® Ultra™ H Directional RNA Library Prep Kit for Illumina (New England Biolabs #E7760L) and the NEBNext Poly(A) mRNA Magnetic Isolation Module (New England Biolabs #E7490) according to the manufacturer's directions. Sequencing was performed on an Illumina Novaseq SP Paired-End 150 bp format.

RNA-Seq Data Analysis

The RNA-Seq data was analyzed using Basepair software (https://www.basepairtech.com/) with a pipeline that included the following steps. Reads were aligned to the transcriptome derived from UCSC genome assembly hg19 using STAR (Dobin, A. et al. Bioinformatics 2013 29:5-21) with default parameters. Read counts for each transcript were measured using featureCounts (Liao, Y., et al. Bioinformatics 2014 30:923-930). Differentially expressed genes were determined using DESeq2 (Love, M I., et al. Genome Biol. 2014 15, 550) and a cut-off of 0.05 on adjusted p-value (corrected for multiple hypotheses testing) was used for creating lists and heatmaps, unless otherwise stated. GSEA was performed on normalized gene expression counts, using gene permutations for calculating p-value.

Ingenuity Pathway Analysis (IPA)

Pathway analysis of differentially expressed genes was performed using Ingenuity Pathway Analysis software (Qiagen). Core analyses were performed using filtered differential expression data (±0.58 log 2 fold change and adjusted p-value 0.05).

Primary Neuronal Cultures

Cortical neurons from embryonic day 18.5 C57BL/6J mouse were prepared following the protocol previously reported (Alzate-Correa, D., et al. Methods Mol. Biol. 2020 2050:145-152), including some modifications. The dissected cortices were dissociated with the Neuronal Tissue Dissociation Kit—Postnatal Neurons (Miltenyi Biotec #130-094-802) and incubated for 20 min at 37gentleMACS Octo Dissociator with Heaters (Miltenyi Biotec #130-096-427). Dissociated cells were resuspended in 5% BSA prepared in PBS and Neurons were isolated from the cell suspension using the Neuron Isolation Kit (Miltenyi Biotec #130-115-389). Isolated neurons were resuspended in neuronal culture media composed of Neurobasal™ (Thermo Fisher Scientific #21103049) supplemented with 2 mM GlutaMAX™ (Thermo Fisher Scientific #35050061) and 2% NeuroCult™ SM1 Neuronal Supplement (Stemcell Technologies #05711). Neurons were plated on poly-D-lysine (Thermo Fisher Scientific #A3890401) coated glass coverslips (Fisher Scientific #1254580) at a density of 130×10³ cell/cm². Primary Neuronal Cultures were exposed to skin derived EVs for the specified hours and then processed for Cell viability analysis, fixed for immunocytochemistry or total RNA extraction for qPCR.

Cell Viability

Cytotoxicity of EVs derived from the skin of B6129SF2/J or 3×Tg-AD mice was evaluated with the LIVE/DEAD Viability/Cytotoxicity Kit for mammalian cells (Thermo Fisher Scientific #L3224) following manufacturer's instructions. Briefly, after day-in-vitro (DIV) 7 primary neuronal cultures were exposed for 24h to EVs from 10 weeks old B6129SF2/J or 3×Tg-AD mice, each well was exposed to 1-3×10⁹ EVs/μL particles. 24 h after exposure neuronal cultures were washed once with sterile PBS followed by incubation at 37° C. for 25 minutes with a PBS solution containing calcein AM (1 uM) and ethidium homodimer-1 (1 um). Finally glass coverslips were fresh mounted using PBS and microphotographs were taken using a Nikon Eclipse 2000 microscope. Cell viability was determined by quantification of the percentage of LIVE (calcein AM positive) and DEAD (ethidium homodimer-1 positive) cells using FIJI ImageJ software (Schindelin, J. et al. Nat. Methods 2012 9:676-682).

Labeled Extracellular Vesicles Uptake

Pelleted EVs were fluorescently labeled using the “PKH26 Red Fluorescent Cell Linker Kit” (Sigma) following the provided protocol. 8 days after seeding onto coverslips, primary neuron cultures were exposed to labeled EVs by replacing 1/3 of media volume with media containing 3×Tg-AD skin-derived EVs at a concentration of 1-3×109 EVs/μL prior to fixation in 10% formalin 24 hours later. Blocking prior to immunocytochemistry was performed using 5% normal goat serum in PBS-T for 90 minutes. The immunocytochemistry was performed using a Tuj1 antibody (Abcam 107216) in 1:250 PBS-T incubated overnight at 4° C. Afterwards, the samples were washed with PBS-T followed by the addition of secondary antibody (Abcam 150173, A=488) at a concentration of 1:200 in PBS-T and finally 1:10,000 DAPI in PBS for 1 minute. The coverslips were then mounted onto slides using Vectashield (Vector Labs #H-1700). The slides were subsequently imaged with immunofluorescence and confocal microscopy using a Nikon Eclipse 200 microscope.

Statistical Analysis

Statistical analyses were conducted using SigmaPlot (version 14) and Graphpad (version 8.4). The qPCR data of skin-derived EVs from 3×Tg-AD (3 to 40 weeks) and 3×Tg-AD/B6129SF2/J (21 day to 23 weeks) were analyzed using one and two way anovas respectively. Data with adjusted p-values <0.05 were considered statistically significant.

### Example 3: Evaluating the mRNA Content of Skin-Derived Extracellular Vesicles for Genetic Biomarkers of Alzheimer's Disease

The purpose of this Example was to identify potential biomarkers of Alzheimer's Disease (AD) through the use of RNA Seq to characterize differences in mRNA expression in skin-derived EVs collected from 3×Tg-AD triple transgenic mouse model of AD and B6129SF2/J controls at 10 and 23 weeks of age (n=3). Reads were aligned to the transcriptome derived from UCSC genome assembly mm10 using STAR (Dobin, A. et al. Bioinformatics 2013 29:15-21) with default parameters. This was followed by differential expression DeSeq2 (Love, M. I., et al. Genome Biol. 2014 15:550) analysis of several different comparison groups of both disease and age including:
3×Tg-AD 23 weeks vs. B6129SF2/J 23 weeks (effects of AD in older mice),
3×Tg-AD 23 weeks vs. 3×Tg-AD 10 weeks (effects of age in AD mice),
3×Tg-AD 10 weeks vs. B6129SF2/J 10 weeks (effects of AD in younger mice), and
B6129SF2/J 23 weeks vs. B6129SF2/J 10 weeks (effects of age in control mice).

The differential expression results were subsequently filtered (p-value 0.05, log 2foldchange±1). Ingenuity Pathway Analysis (IPA, Qiagen) was used to identify overlap between groups, as well as relevant pathways and functions among all groups. In order to identify potential biomarkers, the list of differentially expressed genes in group 1 (effects of AD in older mice) was first examined, showing that any differentially expressed genetic biomarkers would likely be detectable at this point in the 3×Tg-AD model, in which cognitive impairment and the first symptoms of AD have been observed at as early as 5 months (˜22 weeks) of age (Oddo S., et al. Neuron. 2003 39(3):409-21). While genes in this list alone could contain potential biomarkers, the list was further compared for overlap with group 2 (effects of age in AD mice) to see if any changes in the expression of these genes could be detected between the 10 week and 23 weeks timepoints of AD progression. A list of common genes between these two groups was created, which represents genes that change in expression between weeks 10 and 23 of AD progression that are still detectable at 23 weeks compared with controls. In order to determine how these genes were differentially expressed early on in AD, as well as normal healthy aging, this list of common genes was compared to both group 1 (effects of AD in younger mice) and group 4 (effects of age in control mice) respectively.

Results

Differential expression analysis of each group revealed the following differences in gene expression between each group: Group 1 (240 up/426 down), Group 2 (150 up/143 down), Group 3 (91 up/79 down) and Group 4 (98 up/98 down) (FIG. 9). For a complete list of differentially expressed genes between each condition, see supplemental document 3. Pathway analysis on all four groups revealed the dysregulation of several pathways that occurred as a result of the AD condition including interleukin-6 signaling (IL6), high mobility group box protein 1 signaling (HMGB1), and interleukin-8 signaling (IL8 (FIG. 10A). Examples of some of the top dysregulated disease and function pathways seen in the AD samples include migration of myeloid cells and accumulation of neutrophils (FIG. 10B).

Skin-derived EVs in 3×Tg-AD show differential expression of genes and pathways known to be involved in AD that change as the disease progresses. One prominent example of this is S100A8, which has been previously observed to be upregulated in the AD brain (Cristóvão, J. S. & Gomes, C. M. 2019 Front. Neurosci. 13:463). Furthermore, S100A8 upregulation of expression in the brain precedes amyloid plaque formation in TG2576 mice (Lodeiro M, et al. Gerontol A Biol Sci Med Sci. 2017 72(3):319-328) which express the same amyloid precursor protein mutation as the 3×Tg-AD model. S100A8 was found to be upregulated in 3×Tg-AD skin-derived EVs as a function of age/disease progression, while the opposite was observed in control mice as they age. This indicates that S100A8 could be a potential biomarker found within skin-exosomes of AD given its upregulation is unique to AD and not related to aging. Interestingly however, S100A8 was found to be downregulated in the group 4 comparison (effects of AD in younger mice), which suggests this gene may be down regulated in skin EVs earlier on in disease progression. In addition, a related gene that is also involved in AD, S100A9 (Cristóvão, J. S. & Gomes, C. M. 2019 Front. Neurosci. 13:463), was found to be upregulated in group 1, but not group 2. Another interesting gene found to be upregulated in group 1 was Mitogen-Activated Protein Kinase 13 (MAPK13). MAPK13 has been identified as a major kinase that phosphorylates tau epitopes related to AD neurofibrillary tangles (Cavallini A., et al. J Biol Chem. 2013 288(32):23331-23347). In the group 4 comparison of aged controls vs. young controls, MAPK13 expression showed the opposite trend and was downregulated. Several other differentially expressed genes found in the comparison of group 1 and 2 were also found to be related to AD, such as fatty acid amide hydrolase (D'Addario C., et al. PLoS One. 2012 7(6):e39186) and activity regulated cytoskeleton associated protein (Perez-Cruz., et al. J Neurosci. 2011 31(10):3926-34).

Pathway analysis on all groups implicated dysregulation of pathways related to known components of AD in group 1, such as calcium signaling (Tong B C, et al. Biochim Biophys Acta Mol Cell Res. 2018 1865(11 Pt B):1745-1760), HMGB1 signaling (Paudel, Y. N., et al. Frontiers in Neuroscience, 2018 12), IL-6 (Cojocaru I M, et al. Rom J Intern Med. 2011 49(1):55-58) signaling, and IL-8 signaling (Ryu, J. K., et al. J Neuroinflammation 2015 12:144) (FIG. 10A). The IPA data also suggested the increase of pathways related to immune and inflammatory responses including: accumulation of neutrophils, which is linked to AD as well as the activities of IL-6 and IL-8 (Park J, et al. Front Immunol. 2019 10:2231) (FIG. 10B).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for diagnosing a neurodegenerative disease in a subject, comprising isolating exosomes from the subject and assaying the exosomes for the presence of one or more biomarkers of a neurodegenerative disease, or dysregulation of one or more pathways associated with a brain disorder, disease, or injury.

2-10. (canceled)

11. A method for treating a subject with a brain disease, disorder, or injury, comprising delivering intracellularly into skin cells of the subject a polynucleotide comprising nucleic acid sequences encoding therapeutic genes to produce skin-derived exosomes containing the therapeutic gene or gene expression product thereof.

12. The method of claim 11, wherein the brain disease is a neurodegenerative disease.

13. The method of claim 12, wherein the neurodegenerative disease comprises Alzheimer's Disease (AD), Parkinson's disease (PD), amyotrophic lateral sclerosis (ALS), and Huntington's disease (HD).

14. The method of claim 11, wherein the brain disease is a brain cancer.

15. The method of claim 11, wherein the brain disease is a stroke or ischemia.

16. The method of claim 11, wherein the brain injury is a traumatic brain injury.

17. The method of claim 11, wherein the therapeutic gene is a vasculogenic factor.

18. The method of claim 17, wherein the vasculogenic factor comprises Etv2, Fli1, VEGFA, VEGFB, VEGFC, VEGFD, bFGF, Sox17, Oct4, Klf4, or any combination thereof.

19. The method of claim 11, wherein the therapeutic gene is a neurogenic factor.

20. The method of claim 17, wherein the neurogenic factor is selected from the group consisting of Ascl1, Ascl2, Ascl3, Ascl5, Neurog1, Neurog2, Neurog3, Neurod1, Neurod2, Neurod4, Neurod6, Atoh1, Atoh7, Atoh8, Myf5, Ptf1a, Brn3c, Brn3a, Brn3b, Brn1, Brn2, Brn4, Oct4, Oct6, Pit1, Brn5, Myt11, and Nurr1, or any combination thereof.

21. The method of claim 11, wherein the therapeutic gene is an siRNA or miRNA that inhibits APP.

22. The method of claim 21, wherein the miRNA is selected from the group consisting of hsa-miR-106b-5p, hsa-miR-101-3p, hsa-miR-520c-3p, hsa-miR-106a-5p, hsa-miR-20a-5p, hsa-miR-17-5p, hsa-miR-15a-5p, hsa-miR-130a-3p, hsa-let-7d-5p, hsa-let-7a-5p, hsa-miR-16-5p, hsa-miR-144-3p, hsa-miR-4422, hsa-let-7f-1-3p, hsa-let-7a-3p, hsa-let-7b-3p, hsa-miR-98-3p, hsa-miR-380-3p, hsa-miR-6835-3p, hsa-miR-4772-5p, hsa-miR-101-3p, hsa-miR-4719, hsa-miR-520f-3p, hsa-miR-3908, hsa-miR-4269, hsa-miR-323a-3p, hsa-miR-6715b-5p, hsa-miR-153-3p, hsa-miR-4495, hsa-miR-4786-5p, hsa-miR-3911, hsa-miR-6085, and hsa-miR-6813-5p, or any combination thereof.

23. The method of claim 11, wherein the therapeutic gene is an siRNA or miRNA that inhibits MAPT.

24. The method of claim 23, wherein the miRNA is selected from the group consisting of hsa-miR-34c-5p, hsa-miR-657, hsa-miR-4728-5p, and hsa-miR-3978, or any combination thereof.

25. The method of claim 11, wherein the therapeutic gene is an anti-Inflammatory gene.

26. The method of claim 25, wherein the anti-Inflammatory gene is selected from the group consisting of PPARγ, II-10, II-27, Trem2, and IkBα, and Sirt1, or any combination thereof.

27. The method of claim 11, wherein the therapeutic gene is an siRNA or miRNA that targets a pro-inflammatory gene.

28. The method of claim 27, wherein the pro-inflammatory gene is P2X₄R, TLR4, CX3CR1, or IL-1β.

29. A method for treating a neurological disease in a subject, comprising administering to the skin of the subject an agent that inhibits exosomal release from the skin to reducing trafficking to the brain.

30. (canceled)
